(19) United States
(12) Patent Application Publication
(10) Pub. No.: US 2003/0045499 A1
(43) Pub. Date: Mar. 6, 2003

(54) Dendritic cells transduced with a wild-type self gene elicit potent antitumor immune responses

(75) Inventors: Dmitry Gabrilovich, Aurora, IL (US); David Carbone, Franklin, TN (US); Sunil Chada, Missouri City, TX (US); Abner Mhashilkar, Houston, TX (US)

Correspondence Address: Robert E. Hanson, FULBRIGHT & JAWORSKI L.L.P., Suite 2400, 600 Congress Avenue, Austin, TX, 78701, US

(73) Assignee: Vanderbilt University and Introgen Therapeutics, Inc.

(21) Appl. No.: 10/216,346

(22) Filed: Aug. 9, 2002

Related U.S. Application Data
(62) Division of application No. 09/526,320, filed on Mar. 15, 2000.
(60) Provisional application No. 60/124,482, filed on Mar. 15, 1999.
(60) Provisional application No. 60/124,388, filed on Mar. 15, 1999.

(51) Int. Cl. A61K 48/00

(52) U.S. Cl. 514/44

(57) ABSTRACT

The present invention relates to immunotherapy methods for treating hyperproliferative disease or pathogen-induced diseases in humans. More specifically, the invention is directed, in one embodiment, to methods for treating a subject with a hyperproliferative disease in which the expression of a self gene is upregulated in hyperproliferative cells. In another embodiment, an adenoviral expression construct comprising a self gene under the control of a promoter operable in eukaryotic cells is intradermally administered to said hyperproliferative cells. In another embodiment of the present invention, a pathogen-induced disease in which the pathogen gene expression is increased or altered, is treated by intradermally administered a pathogen gene under the control of a promoter operable in eukaryotic cells. The present invention thus provides immunotherapies for treating hyperproliferative and pathogen diseases by attenuating the natural immune systems CTL response against hyperproliferative cells or overexpressing mutant p53 antigens.

### BACKGROUND OF THE INVENTION

The present application claims the benefit of U.S. Provisional Application Serial Number 60/124,482 and U.S. Provisional Application Serial Number 60/124,388, both of which were filed on Mar. 15, 1999. The government owns rights in the present invention pursuant to grant number CA61242 from the National Cancer Institute.

A. FIELD OF THE INVENTION

The present invention relates generally to the fields of immunology and cancer therapy. More particularly, it concerns a method of eliciting a cytotoxic T lymphocyte response directed against self gene antigens presented by hyperproliferative cells.

B. DESCRIPTION OF RELATED ART

Normal tissue homeostasis is a highly regulated process of cell proliferation and cell death. An imbalance of either cell proliferation or cell death can develop into a cancerous state (Solyanik et al., 1995; Stokke et al., 1997; Mumby and Walter, 1991; Natoli et al., 1998; Magi-Galluzzi et al., 1998). For example, cervical, kidney, lung, pancreatic, colorectal and brain cancer are just a few examples of the many cancers that can result (Erlandsson, 1998; Kolmel, 1998; Mangray and King, 1998; Gertig and Hunter, 1997; Mougin et al., 1998). In fact, the occurrence of cancer is so high, that over 500,000 deaths per year are attributed to cancer in the United States alone.

The maintenance of cell proliferation and cell death is at least partially regulated by proto-oncogenes. A proto-oncogene can encode proteins that induce cellular proliferation (e.g., sis, erbB, src, ras and myc), proteins that inhibit cellular proliferation (e.g., Rb, p53, NF1 and WT1) or proteins that regulate programmed cell death (e.g., bcl-2) (Ochi et al., 1998; Johnson and Hamdy, 1998; Liebermann et al., 1998). However, genetic rearrangements or mutations to these proto-oncogenes, results in the conversion of a proto-oncogene into a potent cancer causing oncogene. Often, a single point mutation is enough to transform a proto-oncogene into an oncogene. For example, a point mutation in the p53 tumor suppressor protein results in the complete loss of wild-type p53 function (Vogelstein and Kinzler, 1992; Fulchi et al., 1998) and acquisition of “dominant” tumor promoting function.

Currently, there are few effective options for the treatment of many common cancer types. The course of treatment for a given individual depends on the diagnosis, the stage to which the disease has developed and factors such as age, sex and the general health of the patient. The most conventional options of cancer treatment are surgery, radiation therapy and chemotherapy. Surgery plays a central role in the diagnosis and treatment of cancer. Typically, a surgical approach is required for biopsy and to remove cancerous growth. However, if the cancer has metastasized and is widespread, surgery is unlikely to result in a cure and an alternate approach must be taken. Radiation therapy, chemotherapy and immunotherapy are alternatives to surgical treatment of cancer (Mayer, 1998; Ohara, 1998; Ho et al., 1998). Radiation therapy involves a precise aiming of high energy radiation to destroy cancer cells and much like surgery, is mainly effective in the treatment of non-metastasized, localized cancer cells. Side effects of radiation therapy include skin irritation, difficulty swallowing, dry mouth, nausea, diarrhea, hair loss and loss of energy (Curran, 1998; Brizel, 1998).

Chemotherapy, the treatment of cancer with anti-cancer drugs, is another mode of cancer therapy. The effectiveness of a given anti-cancer drug therapy is often limited by the difficulty of achieving drug -delivery throughout solid tumors (el-Kareh and Secomb, 1997). Chemotherapeutic strategies are based on tumor tissue growth, wherein the anti-cancer drug is targeted to the rapidly dividing cancer cells. Most chemotherapy approaches include the combination of more than one anti-cancer drug, which has proven to increase the response rate of a wide variety of cancers (U.S. Pat. No. 5,824,348; U.S. Pat. No. 5,633,016 and U.S. Pat. No. 5,798,339). A major side effect of chemotherapy drugs is that they also affect normal tissue cells, with the cells most likely to be affected being those that divide rapidly (e.g., bone marrow, gastrointestinal tract, reproductive system and hair follicles). Other toxic side effects of chemotherapy drugs are sores in the mouth, difficulty swallowing, dry mouth, nausea, diarrhea, vomiting, fatigue, bleeding, hair loss and infection.

Immunotherapy, a rapidly evolving area in cancer research, is yet another option for the treatment of certain types of cancers. For example, the immune system identifies tumor cells as being foreign and thus are targeted for destruction by the immune system. Unfortunately, the response typically is not sufficient to prevent most tumor growths. However, recently there has been a focus in the area of immunotherapy to develop methods that augment or supplement the natural defense mechanism of the immune system. Examples of immunotherapies currently under investigation or in use are immune adjuvants (e.g., Mycobacterium bovis, Plasmodium falciparum, dinitrochlorobenzene and aromatic compounds) (U.S. Pat. No. 5,801,005; U.S. Pat. No. 5,739,169; Hui and Hashimoto, 1998; Christodoulides et al., 1998), cytokine therapy (e.g., interferons α, β and γ; IL-1, GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Edward and Villaseca, 1998; U.S. Pat. No. 5,830,880 and U.S. Pat. No. 5,846,945) and monoclonal antibodies (e.g., anti-ganglioside GM2, anti-HER-2, anti-p 185) (Pietras et al., 1998; Hanibuchi et al., 1998; U.S. Pat. No. 5,824,311).

As mentioned above, proto-oncogenes play an important role in cancer biology. For example, Rb, p53, NF1 and WT1 tumor suppressors, are essential for the maintenance of the non-tumorogenic phenotype of cells (reviewed by Soddu and Sacchi, 1998). Approximately 50% of all cancers have been found to be associated with mutations of the p53 gene, which result in the loss of p53 tumor suppressor properties (Levine et al., 1991; Vogelstein and Kinzler, 1992; Hartmann et al., 1996a; Hartmann et al., 1996b). Mutations in the p53 gene also result in the prolongation of the p53 half-life in cells and the overexpression of p53 protein. In normal cells, p53 is undetectable due to its high turnover rate. Thus, p53 overexpression in cancerous cells results in multiple immunogenic p53 epitopes which can be used in immunotherapy. The high incidence of cancer related to mutations of the p53 gene has prompted many research groups to investigate p53 as a route of cancer treatment via gene replacement. The proto-oncogenes sis, erbB, src, ras and myc, encoding proteins that induce cellular proliferation, and the proto-oncogenes of the Bcl-2 family that regulate programmed cell death also play important roles in the non-tumorogenic phenotype of cells.

A few also have explored the use of p53 in immunotherapy. For example, in an in vitro assay, p53 mutant peptides capable of binding to HLA-A2.1 and inducing primary cytotoxic T lymphocyte (CTL) responses were identified (Houbiers et al., 1993). In a study in which synthetic p53 mutant and wild-type peptides were screened for immunogenicity in mice, it was observed that only mutant p53 epitopes were capable of eliciting a CTL response (Bertholet et al., 1997). In contrast, the immunization of BALB/c mice with bone marrow-derived dendritic cells (DC) in the presence of GM-CSF/IL-4 and prepulsed with the H-2Kd binding wild-type p53 peptide (232-240) was observed to induce p53 anti-peptide CTL response (Ciemik et al., 1996; Gabrilovich et al., 1996; Yanuck et al., 1993; DeLeo, 1998; Mayordomo et al., 1996). Further, the intradermal and intramuscular injection of naked plasmid DNA encoding human wild-type p53 and the intravenous injection of human wild-type p53 presented by a recombinant canarypox vector have been successful in the destruction of tumors (Hurpin et al., 1998).

Despite the foregoing, there currently exist no methods of self gene-based immunotherapy capable of utilizing wild-type self genes to generate an antitumor immune response specific for a variety of cells overexpressing different mutant self proteins. This would permit the treatment of any cancerous or pre-cancerous cell associated with increased or altered expression of the self gene. Further, it would eliminate the need to identify the site of self gene mutation in each patient and generate customized self gene mutant peptides for immunotherapy. Thus, the need exists for an immunotherapy that is capable of attenuating or enhancing the natural immune systems CTL response against hyperproliferative cells with increased or altered expression of mutant self gene antigens.

### SUMMARY OF THE INVENTION

Therefore, there exists a need for an immunotherapy that is capable of augmenting the natural immune systems CTL response against hyperproliferative cells or pathogen infected cells expressing an altered self gene antigen or pathogenic antigen, respectively. The present invention also provides a method of eliciting a cytotoxic T lymphocyte response directed against p53 antigens presented by hyperproliferative cells. In one embodiment of the invention, there is provided a method for treating a subject with a hyperproliferative disease.

The treatment of a hyperproliferative disease in the present invention comprises the steps of identifying a subject with a hyperproliferative disease, characterized by alteration or increased expression of a self gene product in at least some of the hyperproliferative cells in the patient. Following identification of a subject with a hyperproliferative disease, an expression construct comprising a self gene under the control of a promoter operable in eukaryotic dendritic cells is intradermally administered to the subject. The self gene product is expressed by dendritic cells and presented to immune effector cells, thereby stimulating an anti-self gene product response.

In one embodiment, the self-gene product is an oncogene, wherein the oncogene may be selected from the group consisting of tumor suppressors, tumor associated genes, growth factors, growth-factor receptors, signal transducers, hormones, cell cycle regulators, nuclear factors, transcription factors and apoptic factors. In preferred embodiments, the tumor suppressor is selected from the group consisting of Rb, p53, p16, p19, p21, p73, DCC, APC, NF-1, NF-2, PTEN, FHIT, C-CAM, E-cadherin, MEN-I, MEN-II, ZAC1, VHL, FCC, MCC , PMS1, PMS2, MLH-1, MSH-2, DPC4, BRCA1, BRCA2 and WT-1. In preferred embodiments, the tumor suppressor is p53. In preferred embodiments, the growth-factor receptor is selected from the group consisting of FMS, ERBB/HER, ERBB-2/NEU/HER-2, ERBA, TGF-β receptor, PDGF receptor, MET, KIT and TRK. In preferred embodiments, the signal transducer is selected from the group consisting of SRC, AB1, RAS, AKT/PKB, RSK-1, RSK-2, RSK-3, RSK-B, PRAD, LCK and ATM. In preferred embodiments, the transcription factor or nuclear factor is selected from the group consisting of JUN, FOS, MYC, BRCA1, BRCA2, ERBA, ETS, EVII, MYB, HMGI-C, HMGI/LIM, SKI, VHL, WT1, CEBP-β, NFKB, IKB, GL1 and REL. In preferred embodiments, the growth factor is selected from the group consisting of SIS, HST, INT-1/WT1 and INT-2. In preferred embodiments, the apoptic factor is selected from the group consisting of Bax, Bak, Bim, Bik, Bid, Bad, Bcl-2, Harakiri and ICE proteases. In preferred embodiments, the tumor-associated gene is selected from the group consisting of CEA, mucin, MAGE and GAGE.

The expression construct may be a viral vector, wherein the viral vector is an adenoviral vector, a retroviral vector, a vaccinia viral vector, an adeno-associated viral vector, a polyoma viral vector, an alphavirus vector, or a herpesviral vector. In preferred embodiments, the viral vector is an adenoviral vector.

In certain embodiments, the adenoviral vector is replication-defective. In another embodiment, the replication defect is a deletion in the E1 region of the virus. In certain embodiments, the deletion maps to the E1B region of the virus. In other embodiments, the deletion encompasses the entire E1B region of the virus. In another embodiment, the deletion encompasses the entire E1 region of the virus.

In one embodiment of the present invention, the promoter operable in eukaryotic cells may be selected from the group consisting of CMV IE, dectin-1, dectin-2, human CD11c, F4/80 and MHC class II. In preferred embodiments, the promoter is CMV IE. In another embodiment the expression vector further comprises a polyadenylation signal.

It is contemplated, in one embodiment of the present invention, that the hyperproliferative disease is cancer, wherein the cancer may be selected from the group consisting of lung, head, neck, breast, pancreatic, prostate, renal, bone, testicular, cervical, gastrointestinal, lymphoma, brain, colon, skin and bladder. In other embodiments, the hyperproliferative disease is non-cancerous and may be selected from the group consisting of rheumatoid arthritis (RA), inflammatory bowel disease (IBD), osteoarthritis (OA), pre-neoplastic lesions in the lung and psoriasis.

In other embodiments, the subject treated for a hyperproliferative disease is a human. It is contemplated in certain embodiments administering to the subject at least a first cytokine selected from the group consisting GM-CSF, IL-4, C-KIT, Steel factor, TGF-β, TNF-α and FLT3 ligand. In yet another embodiment, a second cytokine, different from the first cytokine, is administered to the subject. In another embodiment, the cytokine is administered as a gene encoded by the expression construct. In other embodiments, the immune effector cells are CTLs.

Also contemplated in the present invention is intradermal administration of the expression construct by a single injection or multiple injections. In one embodiment, the injections are performed local to a hyperproliferative or tumor site. In another embodiment, the injections are performed regional to a hyperproliferative or tumor site. In still another embodiment, the injections are performed distal to a hyperproliferative or tumor site. It is further contemplated, that the injections are performed at the same time, at different times or via continuous infusion.

The present invention comprises a method for inducing a p53-directed immune response in a subject comprising the steps of obtaining dendritic cells from a subject, infecting the dendritic cells with an adenoviral vector comprising a p53 gene under the control a promoter operable in eukaryotic cells and administering the adenovirus-infected dendritic cells to the subject, whereby p53 expressed in the dendritic cells is presented to immune effector cells, thereby stimulating an anti-p53 response.

In another aspect of the present invention, there is provided a method for treating a pathogen-induced disease in a subject comprising the steps of identifying a subject with a pathogen-induced disease characterized by alteration or increased expression of a pathogen gene product in at least some of the pathogen-induced cells in the patient and intradermally administering to the subject an expression construct comprising a pathogen gene under the control of a promoter operable in eukaryoticdendritic cells, whereby the pathogen gene product is expressed by dendritic cells and presented to immune effector cells, thereby stimulating an anti-pathogen gene product response. In one embodiment, the dendritic cells are obtained from peripheral blood progenitor cells. In another embodiment, multiple injections of adenovirus-infected dendritic cells is contemplated.

In one embodiment of the present invention, the pathogen may be selected from the group consisting of bacterium, virus, fungus, parasitic worm, amoebae and mycoplasma. In certain embodiments, the bacterium may be selected from the group consisting of richettsia, listeria and histolytica. In other embodiments the virus may be selected from the group consisting of HIV, HBV, HCV, HSV, HPV, EBV and CMV. In yet another embodiment, the fuingus may be selected from the group consisting of hitoplasma, coccidis, immitis, aspargillus, actinomyces, blastomyces, candidia and streptomyces.

In certain embodiments for the treatment of a pathogen-induced disease, the expression construct is a viral vector and may be selected from the group consisting of an adenoviral vector, a retroviral vector, a vaccinia viral vector, an adeno-associated viral vector, a polyoma viral vector, an alphavirus vector, or a herpesviral vector. In a preferred embodiment, the viral vector is an adenoviralvector, wherein said adenoviral vector is replication-defective. In one embodiment, the replication defect is a deletion in the E1 region of the virus. In other embodiments, the deletion maps to the E1B region of the virus. In yet other embodiments, the deletion encompasses the entire E1B region of the virus. In still other embodiments, the deletion encompasses the entire E1 region of the virus.

The promoter operable in eukaryotic cells may be selected from the group consisting of CMV IE, dectin-1, dectin-2, human CD11c, F4/80 and MHC class II. In preferred embodiments, the promoter is CMV IE. In certain embodiments, the expression vector further comprises a polyadenylation signal.

It is contemplated in embodiments where the expression construct is delivered intradermally, that administration may be by injection. In other embodiments, intradermal administration comprises multiple injections. It is contemplated in the present invention, that the injections are performed local, regional or distal to the pathogen-induced disease site.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Expression of p53 protein in DC infected with Ad-p53. DCs generated from bone marrow were infected with 100 MOI Ad-c or Ad-p53 for 48 h, washed, fixed, permeablized and stained with anti-p53 antibody and analyzed. Non-specific staining—Ad-p53 infected DCs stained only with secondary antibody. Ad-c and Ad-p53, DC infected with corresponding virus stained with anti-p53 antibody. Typical results of one of three studies performed are shown.

FIG. 2A, FIG. 2B and FIG. 2C. Ad-p53 transduced DCs induce anti-p53 immune responses. FIG. 2A. CTL response. Mice were immunized twice with DC infected with either Ad-c (Ad-c DC) or with Ad-p53 (Ad-p53 DC) (iv injections). Ten days after the last immunization, T cells from these mice were restimulated with Ad-p53 DC and a CTL assay was performed. P815-Ad and P815-Ad-p53 targets were prepared by overnight incubation of P815 cells with adenovirus at MOI 100 pfu/ml. Mean±SE of cytotoxicity from four studies is shown. FIG. 2B. CTL responses against MethA mouse tumor sarcoma cells (expressing mutant mouse p53). Mice were immunized, T cells were restimulated and CTL assay was performed exactly as described in FIG. 2A. Target MethA sarcoma cells were pre-incubated with 50 U/ml IFNγ for three days prior the assay. Two studies with the same results were performed. FIG. 2C. T cell proliferation. Mice were immunized as described in FIG. 2A. T cells were isolated and cultured in triplicates with either control untreated DC, Ad-c DC or Ad-p53 DC. ³II-thymidine uptake was measured on day 3. Mean±SE of thymidine incorporation from two studies is shown.

FIG. 3. Immunization with Ad-p53 protects from tumor challenge. Mice were immunized as described in FIG. 2A. Ten days after the second immunization, mice were challenged with 2×10⁵ D459 (mouse cell expressing human p53) cells or with 6×10⁵ MethA sarcoma cells. In studies with D459 cells, each group included 20 mice, in studies with MethA sarcoma they included 11 mice. Differences between groups were statistically significant (p<0.05).

FIG. 4. Treatment with Ad-p53 DC slowed the growth of established tumors. 2×10⁵ D459 cells were inoculated sc into the shaved backs of mice. Treatment with 2×10⁵ Ad-c or Ad-p53 DC was initiated when tumor became palpable (day 5). DC were injected on day 5, 9 and 13. Mice in the control group were sacrificed on day 31 due to bulky tumors, mice that received treatment with Ad-p53 DC were sacrificed on day 49. Ten mice per group were treated. Mean±SE is shown.

### DESCRIPTION OF ILLUSTRATIVE EMBODIMENTS

The present invention contemplates the treatment of hyperproliferative disease by identifying patients with a hyperproliferative disease in which self gene expression is increased or altered in these hyperproliferative cells. The treatment of such a hyperproliferative disease in one embodiment involves the intradermal administration of a p53 expression construct to dendritic cells, which subsequently present the processed p53 wild-type antigens to immune effector cells. The immune effector cells then mount an anti-p53 response, resulting in the destruction or lysis of hyperproliferative cells presenting mutant p53 antigen. In another embodiment, dendritic cells are obtained from a patient in which p53 expression is upregulated in hyperproliferative cells. The dendritic cells obtained are infected with an adenoviral vector comprising a p53 gene and the p53 adenovirus-infected dendritic cells are administered to the patient. It is contemplated that infected dendritic cells will present self gene antigens to immune effector cells, stimulate an anti-self gene response in the patient and result in the destruction or lysis of hyperproliferative cells presenting mutant self gene antigen.

A. Hyperproliferative Disease

Cancer has become one of the leading causes of death in the Western world, second only behind heart disease. Current estimates project that one person in three in the U.S. will develop cancer, and that one person in five will die from cancer. Cancers can be viewed from an immunologic perspective as altered self cells, that have lost the normal growth-regulating mechanisms.

There are currently three major categories of oncogenes, reflecting their different activities. One category of oncogenes encode proteins that induce cellular proliferation. A second category of oncogenes, called tumor-suppressors genes or anti-oncogenes, function to inhibit excessive cellular proliferation. The third category of oncogenes, either block or induce apoptosis by encoding proteins that regulate programmed cell death.

In one embodiment of the present invention, the treatment of hyperproliferative disease involves the intradermal administration of a self gene expression construct to dendritic cells. It is contemplated that the dendritic cells present the processed self gene wild-type antigens to immune effector cells, which mount an anti-self gene response, resulting in the destruction or lysis of hyperproliferative cells presenting mutant self antigen. The three major categories of oncogenes are discussed below and listed in Table 1.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene is a secreted growth factor. Oncogenes rarely arise from genes encoding growth factors, and at the present, sis is the only known naturally occurring oncogenic growth factor.

The proteins fms, erbA, erbB and neu are growth factor receptors. Mutations to these receptors result in loss of regulatable function. For example, a point mutation affecting the transmembrane domain of the nue receptor protein results in the nue oncogene. The erbA oncogene is derived from the intracellular receptor for thyroid hormone. The modified oncogenic erbA receptor is believed to compete with the endogenous thyroid hormone receptor, causing uncontrolled growth.

The largest class of oncogenes are the signal transducing proteins (e.g., src, abl and ras) are signal transducers. The protein src, is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins jun, fos and myc are proteins that directly exert their effects on nuclear functions as transcription factors. Table 1 lists a variety of the oncogenes described in this section and many of those not described.

2. Inhibitors of Cellular Proliferation

The tumor suppressor oncogenes function to inhibit excessive cellular proliferation. The inactivation of these genes results destroys their inhibitory activity, resulting in unregulated proliferation. The tumor suppressors p53, p16 and C-CAM are described below.

High levels of mutant p⁵³ have been found in many cells transformed by chemical carcinogenesis, ultraviolet radiation, and several viruses. The p53 gene is a frequent target of mutational inactivation in a wide variety of human tumors and is already documented to be the most frequently-mutated gene in common human cancers. It is mutated in over 50% of human NSCLC (Hollstein et al., 1991) and in a wide spectrum of other tumors. A variety of cancers have been associated with mutations of the p53 gene, which result in the loss of p53 tumor suppressor properties. Mutations in the p53 gene further account for approximately 50% of all cancers that develop (Vogelstein and Kinzler, 1992; Levine et al., 1991), with the majority of these mutations being single-base missense mutations (Kovach et al., 1996). It has been observed that mutations resulting in a loss of p53 function also result in high nuclear and cytoplasmic concentrations (i.e. overexpression) of mutant p53 protein (Oldstone et al., 1992; Finlay et al., 1988). In contrast, functional wild-type p53 protein is expressed at very low levels in cells.

The high cellular concentrations of p53 mutant protein has recently received much attention as an avenue for cancer immunotherapy. The general concept is to elicit an immune response against tumor cells presenting mutant p53 peptides bound to MHC molecules on the cell surface. The generation of an anti-tumor response using mutant p53 peptides as antigens has been demonstrated in several studies (McCarty et al., 1998; Gabrilovich-et al., 1996; Mayordomo et al., 1996; Zitvogel et al., 1996) However, this approach to cancer immunotherapy has several limitations. For example, p53 mutations can occur at many different sites in the protein, making it necessary to identify the site of the mutation in each patient before creating a specific mutant peptide for p53 cancer therapy. Further, not all mutations are contained in regions of the protein known to bind to MHC molecules, and therefore would not elicit an anti-tumor response (DeLeo, 1998).

The limitations described above have stimulated the search for antigenic epitopes in wild-type p53 sequences common to the vast majority of tumor derived p53 proteins. Wild-type p53 peptide-specific cytotoxic T lymphocytes have been produced from human and murine responding lymphocytes, some of which recognized p53-overexpressing tumors in vitro and in vivo (Theobald, et al., 1995; Ropke et al., 1996; Nijman et al., 1994; U.S. Pat. No. 5,747,469, specifically incorporated herein by reference in its entirety). However, since the presentation of antigens is MHC class I restricted, only certain peptides can successfully be administered in certain patients, due to the polymorphic nature of the MHC class I peptide binding site. Further, it is not practical to identify all possible p53 peptides binding to a particular individuals repertoire of MHC molecules. Additionally, a peptide vaccine that does bind to a patient's class I MHC may not be sufficiently presented by MHC class II, the molecules crucial in the induction of CD4⁺ T cell immune responses.

Researchers have to attempted to identify multiple p53 epitopes, which should permit more effective immune responses against tumor cells expressing multiple p53 genes with mutations at different sites. This could be accomplished by immunizing cells with intact wild-type p53 to take advantage of the overexpression of the whole p53 polypeptide in most human tumors. The dendritic cell (DC) is the cell type best suited for vaccine antigen delivery (described further in section .B), as they are the most potent antigen presenting cells, effective in the stimulation of both primary and secondary immune responses (Steinman, 1991; Celluzzi and Falo, 1997). It is contemplated in the present invention that the transduction of dendritic cells with wild-type p53 protein, using a viral expression construct, will elicit a potent antitumor immune response specific for a variety of cells expressing different mutant p53 proteins. Further, since most mutations of p53 are single-base missense mutations, the approach of the present invention overcomes the limitations of identifying the site of the p53 mutation and subsequent preparation of a customized mutant peptide for immunotherapy. Thus, the method of the present invention provides the basis for a simple and novel approach to immunotherapy based cancer treatment.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p₂₁^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

C-CAM is expressed in virtually all epithelial cells (Odin and Obrink, 1987). C-CAM, with an apparent molecular weight of 105 kD, was originally isolated from the plasma membrane of the rat hepatocyte by its reaction with specific antibodies that neutralize cell aggregation (Obrink, 1991). Recent studies indicate that, structurally, C-CAM belongs to the immunoglobulin (Ig) superfamily and its sequence is highly homologous to carcinoembryonic antigen (CEA) (Lin and Guidotti, 1989). Using a baculovirus expression system, Cheung et al. (1993) demonstrated that the first Ig domain of C-CAM is critical for cell adhesive activity.

Cell adhesion molecules, or CAM's are known to be involved in a complex network of molecular interactions that regulate organ development and cell differentiation (Edelman, 1985). Recent data indicate that aberrant expression of CAM's maybe involved in the tumorigenesis of several neoplasms; for example, decreased expression of E-cadherin, which is predominantly expressed in epithelial cells, is associated with the progression of several kinds of neoplasms (Edelman and Crossin, 1991; Frixen et al., 1991; Bussemakers et al., 1992; Matsura et al., 1992; Umbas et al., 1992). Also, Giancotti and Ruoslahti (1990) demonstrated that increasing expression of α₅β₁ integrin by gene transfer can reduce tumorigenicity of Chinese hamster ovary cells in vivo. C-CAM now has been shown to suppress tumors growth in vitro and in vivo.

Other tumor suppressors that may be employed according to the present invention include RB, APC, DCC, NF-1, NF-2, WT-1, MEN-I, MEN-II, zacl, p73, VHL, MMAC1, FCC and MCC (see Table 1).

3. Regulators of Programmed Cell Death

Apoptosis, or programmed cell death, is an essential occurring process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Mcl-l, Al, Bfl-1) or counteract Bcl-2 function and promote cell death (e.g., Bax, Bak, Bik, Bim, Bid, Bad, Harakiri).

TABLE 1
ONCOGENES

| | | | |
|---|---|---|---|
| Gene | Source | Human Disease | Function |

Growth Factors¹

| | | |
|---|---|---|
| HST/KS | Transfection | FGF family member |
| INT-2 | MMTV promoter | FGF family member |
| | insertion | |
| INTI/WNTI | MMTV promoter | Factor-like |
| | insertion | |
| SIS | Simian sarcoma | PDGF B |
| | virus | |

Receptor Tyrosine Kinases^{1,2}

| | | | |
|---|---|---|---|
| ERBB/HER | Avian erythro- | Amplified, | EGF/TGF-α/ |
| | blastosis virus; | deleted squamous | amphiregulin/ |
| | ALV promoter | cell cancer; | hetacellulin receptor |
| | insertion; | glioblastoma | |
| | amplified human | | |
| | tumors | | |
| ERBB-2/ | Transfected from | Amplified breast, | Regulated by NDF/ |
| NEU/HER-2 | rat glioblatoms | ovarian, gastric | heregulin and EGF- |
| | | cancers | related factors |
| FMS | SM feline | | CSF-1 receptor |
| | sarcoma virus | | |
| KIT | HZ feline | | MGF/Steel receptor |
| | sarcoma virus | | hematopoieis |
| TRK | Transfection | | NGF (nerve growth |
| | from human | | factor) receptor |
| | colon cancer | | |
| MET | Transfection | | Scatter factor/HGF |
| | from human | | receptor |
| | osteosarcoma | | |
| RET | Translocations | Sporadic thyroid | Orphan receptor Tyr |
| | and point | cancer; familial | kinase |
| | mutations | medullary thyroid | |
| | | cancer; multiple | |
| | | endocrine | |
| | | neoplasias 2A and | |
| | | 2B | |
| ROS | URII avian | | Orphan receptor Tyr |
| | sarcoma virus | | kinase |
| PDGF | Translocation | Chronic | TEL (ETS-like |
| receptor | | myclomonocytic | transcription factor)/ |
| | | leukemia | PDGF receptor gene |
| | | | fusion |
| TGF-β | | Colon carcinoma | |
| receptor | | mismatch | |
| | | mutation target | |

NONRECEPTOR TYROSINE KINASES¹

| | | | |
|---|---|---|---|
| ABI | Abelson Mul. V | Chronic | Interact with RB, |
| | | myelogenous | RNA polymerase, |
| | | leukemia | CRK, CBL |
| | | translocation | |
| | | with BCR | |
| FPS/FES | Avian Fujinami | | |
| | SV; GA FeSV | | |
| LCK | Mul. V (murine | | Src family; T cell |
| | leukemia virus) | | signaling; interacts |
| | promoter | | CD4/CD8 T cells |
| | insertion | | |
| SRC | Avian Rous | | Membrane- |
| | sarcoma virus | | associated Tyr |
| | | | kinase with |
| | | | signaling function; |
| | | | activated by |
| | | | receptor kinases |
| YES | Avian Y73 | | Src family; |
| | virus | | signaling |

SER/THR PROTEIN KINASES¹

| | | |
|---|---|---|
| AKT | AKT8 murine | Regulated by |
| | retrovirus | PI(3)K?; regulate |
| | | 70-kd S6 k? |
| MOS | Maloney murine | GVBD; cystostatic |
| | SV | factor; MAP kinase |
| | | kinase |
| PIM-1 | Promoter | |
| | insertion mouse | |
| RAF/MIL | 3611 murine SV; | Signaling in RAS |
| | MH2 avian SV | pathway |

MISCELLANEOUS CELL SURFACE¹

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | suppressor | | homotypic binding; |
| | | | intracellular |
| | | | interacts with |
| | | | catenins |
| PTC/NBCCS | Tumor suppressor | Nevoid basal cell | 12 transmembrane |
| | and Drosophilia | cancer syndrome | domain; signals |
| | homology | (Gorline | through Gli |
| | | syndrome) | homogue CI to |
| | | | antagonize |
| | | | hedgehog pathway |
| TAN-1 | Translocation | T-ALL | Signaling? |
| Notch | | | |
| homologue | | | |

MISCELLANEOUS SIGNALING^{1,3}

| | | | |
|---|---|---|---|
| BCL-2 | Translocation | B-cell lymphoma | Apoptosis |
| CBL | Mu Cas NS-1 V | | Tyrosine- |
| | | | phosphorylated |
| | | | RING finger interact |
| | | | Ab1 |
| CRK | CT1010 ASV | | Adapted SH2/SH3 |
| | | | interact Ab1 |
| DPC4 | Tumor suppressor | Pancreatic cancer | TGF-β-related |
| | | | signaling pathway |
| MAS | Transfection and | | Possible angiotensin |
| | tumorigenicity | | receptor |
| NCK | | | Adaptor SH2/SH3 |

GUANINE NUCLEOTIDE EXCHANGERS
AND BINDING PROTEINS^{3,4}

| | | | |
|---|---|---|---|
| BCR | | Translocated with | Exchanger; protein |
| | | ABL in CML | kinase |
| DBL | Transfection | | Exchanger |
| GSP | | | |
| NF-I | Hereditary tumor | Tumor suppressor | RAS GAP |
| | suppressor | neurofibromatosis | |
| OST | Transfection | | Exchanger |
| Harvey- | HaRat SV; Ki | Point mutations | Signal cascade |
| Kirsten, | RaSV; Balb- | in many human | |
| N-RAS | MoMuSV; | tumors | |
| | transfection | | |
| VAV | Transfection | | S112/S113; |
| | | | exchanger |

NUCLEAR PROTEINS AND TRANSCRIPTION FACTORS^{1,5-9}

| | | | |
|---|---|---|---|
| BRCA1 | Heritable | Mammary cancer/ | Localization |
| | suppressor | ovarian cancer | unsettled |
| BRCA2 | Heritable | Mammary cancer | Function unknown |
| | suppressor | | |
| ERBA | Avian erythro- | | thyroid hormone |
| | blastosis virus | | receptor |
| | | | (transcription) |
| ETS | Avian E26 virus | | DNA binding |
| EVII | MuLV promotor | AML | Transcription factor |
| | insertion | | |
| FOS | FBI/FBR murine | | 1 transcription |
| | osteosarcoma | | factor with c-JUN |
| | viruses | | |
| GLI | Amplified glioma | Glioma | Zinc finger; cubitus |
| | | | interruptus |
| | | | homologue is in |
| | | | hedgehog signaling |
| | | | pathway; inhibitory |
| | | | link PTC and |
| | | | hedgehog |
| HMGG/LIM | Translocation | Lipoma | Gene fusions high |
| | t(3:12) t(12:15) | | mobility group |
| | | | HMGI-C (XT-hook) |
| | | | and transcription |
| | | | factor LIM or acidic |
| | | | domain |
| JUN | ASV-17 | | Transcription factor |
| | | | AP-1 with FOS |
| MLL/ | Translocation/ | Acute myeloid | Gene fusion of |
| VHRX + | fusion ELL with | leukemia | DNA-binding and |
| ELI/MEN | MLL trithorax- | | methyl transferase |
| | like gene | | MLL with ELI RNA |
| | | | pol II elongation |
| | | | factor |
| MYB | Avian myelo- | | DNA binding |
| | blastosis virus | | |
| MYC | Avian MC29; | Burkitt's | DNA binding with |
| | translocation | lymphoma | MAX partner; |
| | B-cell | | cyclin regulation; |
| | lymphomas; | | interact RB?; |
| | promoter | | regulate apoptosis? |
| | insertion avian | | |
| | leukosis virus | | |
| N-MYC | Amplified | Neuroblastoma | |
| L-MYC | | Lung cancer | |
| REL | Avian retriculo- | | NF-κB family |
| | endotheliosis | | transcription factor |
| | virus | | |
| SKI | Avian SKV770 | | Transcription factor |
| | retrovirus | | |
| VHL | Heritable | Von Hippel- | Negative regulator |
| | suppressor | Landau syndrome | or elongin; trans- |
| | | | criptional elongation |
| | | | complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary | Ataxia- | Protein/lipid kinase |
| | disorder | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group C | |
| | | (predisposition | |
| | | leukemia | |
| FHIT | Fragile site | Lung carcinoma | Histidine triad- |
| | 3p14.2 | | related diadenosine |
| | | | 5′,3″″-P¹p⁴ tetra- |
| | | | phosphate |
| | | | asymmetric |
| | | | hydrolase |
| hMLI/MutL | | HNPCC | Mismatch repair; |
| | | | MutL homologue |
| hMSH2/ | | HNPCC | Mismatch repair; |
| MutS | | | MutS homologue |
| hPMS1 | | HNPCC | Mismatch repair; |
| | | | MutL homologue |
| hPMS2 | | HNPCC | Mismatch repair; |
| | | | MutL homologue |
| INK4/MTS1 | Adjacent INK-4B | Candidate MTS1 | p16 CDK inhibitor |
| | at 9p21; CDK | suppressor and | |
| | complexes | MLM melanoma | |
| | | gene | |
| INK4B/ | | Candidate | p15 CDK inhibitor |
| MTS2 | | suppressor | |
| MDM-2 | Amplified | Sarcoma | Negative regulator |
| | | | p53 |
| p53 | Association with | Mutated >50% | Transcription factor; |
| | SV40 T antigen | human tumors, | checkpoint control; |
| | | including | apoptosis |
| | | hereditary | |
| | | Li-Fraumeni | |
| | | syndrome | |
| PRADI/ | Translocation | Parathyroid | Cyclin D |
| BCL1 | with parathyroid | adenoma; B-CLL | |
| | hormone or IgG | | |
| RB | Hereditary | Retinoblastoma; | Interact cyclin/cdk; |
| | retinoblastoma; | osteosarcoma; | regulate E2F |
| | association with | breast cancer; | transcription factor |
| | many DNA virus | other sporadic | |
| | tumor antigens | cancers | |
| XPA | | xeroderma | Excision repair; |
| | | pigmentosum; | photo-product |
| | | skin cancer | recognition; zinc |
| | | predisposition | finger |

4. Non-cancer Hyperproliferative Diseases

In one embodiment of the present invention, it is contemplated that non-cancer hyperproliferative diseases may treated by administering a self gene expression construct capable of eliciting an anti-self gene response. Some of the hyperproliferative diseases contemplated for treatment in the present invention are psoriasis, rheumatoid arthritis (RA), inflamatory bowel disease (IBD), osteo arthritis (OA) and pre-neoplastic lesions in the lung.

B. Pathogen Induced Disease

In other embodiments of the present invention, a method for treating a pathogen-induced disease in a subject in which pathogen-induced disease is characterized by an alteration or increased expression of a pathogen gene product in at least some of the pathogen-induced cells is contemplated. Following identification of altered or increased expression of a pathogen gene product, an expression construct comprising a pathogen gene under the control a promoter operable in eukaryotic cells is intradermally administering to the subject. It is contemplated that the pathogen gene product expressed in the dendritic cells is presented to immune effector cells, stimulating an anti-pathogen gene product response.

In another embodiment, it is contemplated that pathogen such as bacterium, virus, fungus, parasitic worm, amoebae and mycoplasma can be treated using the method of the present invention. For example, anti-pathogen responses to bacteria such as richettsia, listeria and histolytica, viri such as HIV, HBV, HCV, HSV, HPV, EBV and CMV, and fungi such as hitoplasma, coccidis, immitis, aspargillus, actinomyces, blastomyces, candidia and streptomyces, are contemplated in the present invention.

C. Immunologic Responses Related to Self Gene Tumorogenicity

In one embodiment of the present invention, hyperproliferative disease in which p53 expression is upregulated in the hyperproliferative cells is treated by administering a p53 expression construct capable of eliciting an anti-p53 response. Following delivery of the p53 expression construct to a given antigen presenting cell, a cascade of immunologic events must ensue to stimulate the desired anti-p53 response. Thus, a basic understanding of the immunologic responses related to p53 expressionand more generally, self gene expression in hyperproliferative disease is necessary.

1. Cttotoxic T Lymphocytes

T lymphocytes arise from hematopoietic stem cells in the bone marrow, and migrate to the thymus gland to mature. T cells express a unique antigen binding receptor on their membrane (T-cell receptor), which can only recognize antigen in association with major histocompatibility complex (MHC) molecules on the surface of other cells. There are at least two populations of T cells, known as T helper cells and T cytotoxic cells. T helper cells and T cytotoxic cells are primarily distinguished by their display of the membrane bound glycoproteins CD4 and CD8, respectively. T helper cells secret various lymphokines, that are crucial for the activation of B cells, T cytotoxic cells, macrophages and other cells of the immune system. In contrast, a T cytotoxic cells that recognizes an antigen-MHC complex proliferates and differentiates into an effector cell called a cytotoxic T lymphocyte (CTL). CTLs eliminate cells of the body displaying antigen, such as virus infected cells and tumor cells, by producing substances that result in cell lysis.

An important aspect of the present invention is the stimulation of a CTL response directed against wild-type self gene antigen. It has been observed that mutations of the p53 gene result in the overexpression of the mutant p53 protein in tumor cells (Harris, 1996), while wild-type p53 is expressed at low levels in normal cells. It has further been demonstrated that wild-type and mutant p53 peptides can stimulate a CTL response against tumor cells expressing p53 antigenic peptides (DeLeo, 1998; Mayordomo et al., 1996). It is contemplated in the present invention that a similar anti-self gene CTL response will be stimulated by immunizing dendritic cells with intact wild-type self gene polypeptide, and thus can be used as a treatment for hyperproliferative disease.

2. Antigen-presenting Cells

Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane.

In a preferred embodiment of the present invention, dendritic cells are the antigen-presenting cells of choice for self gene delivery and antigen presentation. Dendritic cells are the most potent antigen-presenting cells for the initiation of antigen-specific T cell activation (Arthur et al., 1997). They are also excellent candidates for short term culture and a variety of gene transfer methods (e.g., DNA/liposome complexes, electroporation, CaPO4 precipitation, and recombinant adenovirus) (Arthur et al., 1997). Human and mouse dendritic cells have been successfully modified by adenoviral gene transfer (Sonderbye et al., 1998). In this study, an adenovirus (AdLacZ) was used to express intracellular beta-galactosidase (beta-gal) antigen in the dendritic cells, with approximately 40% of the cells transduced with AdLacZ expressing high levels of beta-gal. In addition, the subcutaneous immunization of mouse dendritic cells with the ovalbumin (OVA) peptide induced an OVA-specific CD8+CTL response (Celluzzi and Falo, 1997).

3. Major Histocompatibilty Complex

The major histocompatibility complex (MHC) is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is refereed to as the HLA complex and in mice the H-2 complex. An important aspect of the present invention is the immunization of dendritic cells with the intact wild-type self gene to take advantage of the relative overexpression of the whole self gene molecule in most human tumors. The approach of p53 immunotherapy is contemplated in one embodiment, to overcome previous immunotherapies that immunized animals with mutant p53 peptides as antigens (Gabrilovich et al., 1996; Mayordomo et al., 1996; Zitgovel et al., 1996). Although the approaches above using mutant p53 peptides were effective at generating anti-tumor responses, they have several limitations. For example, p53 mutations and other self genes occur at many sites in the protein, making it necessary to identify the site of mutation in each patient before constructing a customized mutant peptide for therapy. Furthermore, not all mutations are contained in regions of the protein known to bind to MHC molecules. In another study using wild-type 53 peptides, CTLs were generated from human and murine responding lymphocytes, some of which recognized p53 overexpressing tumors in vitro (Theobald et al., 1995; Ropke et al., 1996; Nijman et al., 1994). However, since presentation of antigens is MHC class I restricted, only certain oligopeptides can be used in certain patients, because of the highly polymorphic MHC class I peptide binding site. It is contemplated in the present invention that immunizing dendritic cells with intact, wild-type self gene protein, will generate a variety of self gene antigens for MHC class I presentation and thus effectively stimulate a cytolytic T lymphocyte response.

D. Assays for Self Gene Upregulation or Altered Expression

In one embodiment of the present invention, the identification of a patient with a hyperproliferative disease in which self gene expression is upregulated is desired. In patients with a detected hyperproliferative disease, a sample of the hyperproliferative tissue will be used to assay upregulation. A wide variety of detection methods can be employed in the present invention to detect the self gene status of a cell. There are numerous antibodies to the oncogenic proteins, hence any assay that utilizes antibodies for detection, for example, ELISAs, Western Blotting, immunoassay techniques, etc. are contemplated as useful in the present invention. Alternatively, assays that employ nucleotide probes may be used to identify the presence of self gene, for example, Southern blotting, Northern blotting or PCRTM techniques. All the above techniques are well known to one of skill in the art and could be utilized in the present invention without undue experimentation.

1. Elisas, Immunoassay and Immunohistological Assay.

In a preferred embodiment of the present invention, immunohistological assays are used to detect self gene increased or altered expression in tumor samples (e.g., tissue sections). Exemplary methods of immunohistochemistry assays and immunfluorescence assays have previously been described (U.S. Pat. No. 5,858,723; WO94/11514, specifically incorporated herein by reference in its entirety). Further immunoassays encompassed by the present invention include, but are not limited to those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo. Immunoassays generally are binding assays. Certain preferred immunoassays are the various types of enzyme linked immunosorbent assays (ELISAs) and radioimmunoassays (RIA) known in the art.

In one exemplary ELISA, the anti-self gene antibodies are immobilized on a selected surface, such as a well in a polystyrene microtiter plate, dipstick or column support. Then, a test composition suspected of containing the desired antigen, such as a clinical sample, is added to the wells. After binding and washing to remove non-specifically bound immune complexes, the bound antigen may be detected. Detection is generally achieved by the addition of another antibody, specific for the desired antigen, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

2. Southern and Northern Blotting Techniques

Southern and Northern blotting are commonly used techniques in molecular biology and well within the grasp of one skilled in the art. Southern and Northern blotting samples are obtained from the hyperproliferative tissue. The DNA and RNA from test cells is recovered by gentle cell rupture in the presence of a cation chelator such as EDTA. The proteins and other cell milieu are removed by admixing with saturated phenol or phenol/chloroform and centrifugation of the emulsion. The DNA and RNA is in the upper aqueous phase, it is deproteinised and mixed with ethanol. This solution allows the DNA and RNA to precipitate, the DNA and RNA can then be recover using centrifugation. In the case of RNA extraction, RNAse inhibitors such as DEPC are needed to prevent RNA degradation.

Electrophoresis in agarose or polyacrylamide gels is the most usual way to separate DNA and RNA molecules. Southern blotting will confirm the identity of the self gene encoding DNA. This is achieved by transferring the DNA from the intact gel onto nitrocellulose paper. The nitrocellulose paper is then washed in buffer that has for example, a radiolabelled cDNA containing a sequence complementary to wild-type self gene DNA. The probe binds specifically to the DNA that encodes a region of self gene and can be detected using autoradiography by contacting the probed nitrocellulose paper with photographic film. Self gene -encoding mRNA can be detected in a similar manner by a process known as Northern blotting. For a more detailed description of buffers gel preparation, electrophoresis condition etc., the skilled artisan is referred to Sambrook, 1989.

3. Polymerase Chain Reaction (PCR™)

PCR™ is a powerful tool in modern analytical biology. Short oligonucleotide sequences usually 15-35 bp in length are designed, homologous to flanking regions either side of the self gene sequences to be amplified. The primers are added in excess to the source DNA, in the presence of buffer, enzyme, and free nucleotides. The source DNA is denatured at 95° C. and then cooled to 50-60° C. to allow the primers to anneal. The temperature is adjusted to the optimal temperature for the polymerase for an extension phase. This cycle is repeated 25-40 times.

In particular the present invention uses PCR™ to detect the self gene status of cells. Mutations in the self gene are first detected with Single Strand Conformation Polymorphism (SSCP) which is based on the electrophoretic determination of conformational changes in single stranded DNA molecules induced by point mutations or other forms of slight nucleotide changes. To identify where the mutation is located at within the self gene, each exon is separately amplified by PCR™ using primers specific for the particular exon. After amplification, the PCR™ product is denatured and separated out on a polyacrylamide gel to detect a shift in mobility due to a conformational change which resulted because of a point mutation or other small nucleotide change in the gene. Mutations result in a change in the physical conformation of the DNA as well as change in the electrical charge of the molecule. Thus during electrophoresis when an electrical charge is applied to the molecule, DNA that is slightly different in shape and charge as compared to wild-type will move at a different rate and thus occupy a different position in the gel. After determination of which DNA fragment contains the mutation, the specific nucleotide changes are detected by DNA sequencing of the amplified PCR™ product. Sequencing of linear DNA breaks down the DNA molecule into its individual nucleotides in the order with which they are assembled in the intact molecule. Separation of the individual nucleotides by electrophoresis on a sequencing gel allows detection of individual nucleotide changes compared to wild-type and is used to determine homo- or heterozygocity of a mutation, which is easily distinguished by the appearance of a single or double band in the sequencing gel.

E. Self Gene Delivery

Many types of cancer have been associated with mutations in oncogenes. These mutations typically result in the overexpression of a mutant self gene protein in tumor cells. It has been further demonstrated that wild-type p53 peptide specific cytotoxic T lymphocytes were generated from human and murine responding lymphocytes and recognized p53 overexpressing tumors in vitro (Theobald et al., 1995; Ropke et al., 1996; Nijman et al., 1994). The present invention contemplates the in vivo treatment of hyperproliferative diseases by eliciting an anti-self gene immune response directed against cells presenting self gene antigen on their surface. In certain embodiments of the present invention, an expression construct comprising a self gene under the control of a promoter operable in eukaryotic cells is administered and expressed in dendritic cells in order to prime an immune response against p53.

1. Viral Transformation

a. Adenoviral Infection

One method for delivery of the recombinant DNA involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a recombinant gene construct that has been cloned therein.

The vector comprises a genetically engineered form of adenovirus. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) have disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus vector may be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus growth and manipulation is known to those of skill in the art, and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

b. Retroviral Infection

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, packaging cell lines are available that should greatly decrease the likelihood of recombination (Markowitz et al., 1988; Hersdorffer et al., 1990).

C. AAV Infection

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin, et al., 1984; Laughlin, et al., 1986; Lebkowski, et al., 1988; McLaughlin, et al., 1988), which means it is applicable for use with the present invention. Details concerning the generation and use of rAAV vectors are described in U.S. Pat. No. 5,139,941 and U.S. Pat. No. 4,797,368, each incorporated herein by reference.

Studies demonstrating the use of AAV in gene delivery include LaFace et al. (1988); Zhou et al. (1 993); Flotte et al. (1993); and Walsh et al. (1994). Recombinant AAV vectors have been used successfully for in vitro and in vivo transduction of marker genes (Kaplitt et al., 1994; Lebkowski et al., 1988; Sarnulski et al., 1989; Shelling and Smith, 1994; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus or a member of the herpes virus family) to undergo a productive infection in cultured cells (Muzyczka, 1992). In the absence of coinfection with helper virus, the wild-type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome or from a recombinant plasmid, and a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and an expression plasmid containing the wild-type AAV coding sequences without the terminal repeats, for example pIM45 (McCarty et al., 1991; incorporated herein by reference). The cells are also infected or transfected with adenovirus or plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions or cell lines containing the AAV coding regions and some or all of the adenovirus helper genes could be used (Yang et al., 1994a; Clark et al., 1995). Cell lines carrying the rAAV DNA as an integrated provirus can also be used (Flotte et al., 1995).

d. Other Viral Vectors

Other viral vectors may be employed as constructs in the present invention. Vectors derived from viruses such as vaccinia virus (Ridgeway, 1988; Baichwal and Sugden, 1986; Coupar et al., 1988) and herpesviruses may be employed. They offer several attractive features for various mammalian cells (Friedmann, 1989; Ridgeway, 1988; Baichwal and Sugden, 1986; Coupar et al., 1988; Horwich et al., 1990). Alternatively, Alphavirus vectors and replicons may be employed (Leitner et al., 2000; Caley et al., 1999).

A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and has been sugested that VEE may be an extremely useful vector for immunizations (Caley et al., 1997). It is contemplated in the present invention, that VEE virus may be useful in targeting dendritic cells.

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Non-viral Delivery

In addition to viral delivery of the self gene, the following are additional methods of recombinant gene delivery to a given host cell and are thus considered in the present invention.

a. Electroporation

In certain preferred embodiments of the present invention, the gene construct is introduced into the dendritic cells via electroporation. Electroporation involves the exposure of a suspension of cells and DNA to a high-voltage electric discharge.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

It is contemplated that electroporation conditions for dendritic cells from different sources may be optimized. One may particularly wish to optimize such parameters as the voltage, the capacitance, the time and the electroporation media composition. The execution of other routine adjustments will be known to those of skill in the art.

b. Particle Bombardment

Another embodiment of the invention for transferring a naked DNA construct into cells involves particle bombardment. This method depends on the ability to accelerate DNA-coated microprojectiles to a high velocity allowing them to pierce cell membranes and enter cells without killing them (Klein et al., 1987). The microprojectiles used have consisted of biologically inert substances such as tungsten, platinum or gold beads.

It is contemplated that in some instances DNA precipitation onto met al particles would not be necessary for DNA delivery to a recipient cell using particle bombardment. It is contemplated that particles may contain DNA rather than be coated with DNA. Hence it is proposed that DNA-coated particles may increase the level of DNA delivery via particle bombardment but are not, in and of themselves, necessary.

Several devices for accelerating small particles have been developed. One such device relies on a high voltage discharge to generate an electrical current, which in turn provides the motive force (Yang et al., 1990). Another method involves the use of a Biolistic Particle Delivery System, which can be used to propel particles coated with DNA through a screen, such as stainless steel or Nytex screen, onto a filter surface covered with cells in suspension. The screen disperses the particles so that they are not delivered to the recipient cells in large aggregates. It is believed that a screen intervening between the projectile apparatus and the cells to be bombarded reduces the size of projectile aggregates and may contribute to a higher frequency of transformation by reducing the damage inflicted on the recipient cells by projectiles that are too large.

For the bombardment, cells in suspension are preferably concentrated on filters, or alternatively on solid culture medium. The cells to be bombarded are positioned at an appropriate distance below the macroprojectile stopping plate. If desired, one or more screens are also positioned between the acceleration device and the cells to be bombarded.

In bombardment transformation, one may optimize the prebombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity or either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of primordial germ cells.

Accordingly, it is contemplated that one may wish to adjust various of the bombardment parameters in small scale studies to fully optimize the conditions. One may particularly wish to adjust physical parameters such as gap distance, flight distance, tissue distance and helium pressure. One may also optimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art.

C. Calcium Phosphate Co-precipitation or DEAE-dextran Treatment

In other embodiments of the present invention, the transgenic construct is introduced to the cells using calcium phosphate co-precipitation. Mouse primordial germ cells have been transfected with the SV40 large T antigen, with excellent results (Watanabe et al., 1997). Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and erythroleukemia cells (Gopal, 1985).

d. Direct Microinjection or Sonication Loading

Further embodiments of the present invention include the introduction of the gene construct by direct microinjection or sonication loading. Direct microinjection has been used to introduce nucleic acid constructs into Xenopus oocytes (Harland and Weintraub, 1985), and LTK fibroblasts have been transfected with the thymidine kinase gene by sonication loading (Fechheimer et al., 1987).

e. Liposome Mediated Transformation

In a further embodiment of the invention, the gene construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a gene construct complexed with Lipofectamine (Gibco BRL) or DOTAP-Cholesterol formulations.

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1.

3. Vectors and Regulatory Signals

Vectors of the present invention are designed, primarily, to transform dendritic cells with the self gene under the control of regulated eukaryotic promoters (i.e., inducible, repressable, tissue specific). Also, the vectors usually will contain a selectable marker if, for no other reason, to facilitate their production in vitro. However, selectable markers may play an important role in producing recombinant cells and thus a discussion of promoters is useful here. Table 2 and Table 3 below, list inducible promoter elements and enhancer elements, respectively.

Preferred for use in the present invention is the cytomegalovirus (CMV) promoter. This promoter is commercially available from Invitrogen in the vector pcDNAIII, which is preferred for use in the present invention. Also contemplated as useful in the present invention are the dectin-1 and dectin-2 promoters. Below are a list of additional viral promoters, cellular promoters/enhancers and inducible promoters/enhancers that could be used in combination with the present invention. Additionally any promoter/enhancer combination (as per the Eukaryotic Promoter Data Base EPDB) could also be used to drive expression of structural genes encoding oligosaccharide processing enzymes, protein folding accessory proteins, selectable marker proteins or a heterologous protein of interest.

TABLE 2
Table 1—Inducible Elements

| Element | Inducer | References |
|---|---|---|
| MT II | Phorbol Ester (TFA) | Palmiter et al., 1982; |
| | Heavy metals | Haslinger and Karin, 1985; |
| | | Searle et al., 1985; Stuart |
| | | et al., 1985; Imagawa et al., |
| | | 1987; Karin ®, 1987; |
| | | Angel et al., 1987b; |
| | | McNeall et al., 1989 |
| MMTV | Glucocorticoids | Huang et al., 1981; Lee |
| (mouse mammary | | et al., 1981; Majors and |
| tumor virus) | | Varmus, 1983; Chandler |
| | | et al., 1983; Lee et al., |
| | | 1984; Fonta et al., 1985; |
| | | Sakai et al., 1986 |
| B-Interferon | poly(rI)X | Tavernier et al., 1983 |
| | poly(rc) | |
| Adenovirus 5 E2 | Ela | Imperiale and Nevins, 1984 |
| Collagenase | Phorbol Ester (TPA) | Angle et al., 1987a |
| Stromelysin | Phorbol Ester (TPA) | Angle et al., 1987b |
| SV40 | Phorbol Ester (TFA) | Angel et al., 1987b |
| Murine MX Gene | Interferon, Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | Ela, SV40 Large T | Taylor et al., 1989; Taylor |
| | Antigen | and Kingston, 1990a, b |
| Proliferin | Phorbol Ester-TPA | Mordacq and Linzer, 1989 |
| Tumor Necrosis | FMA | Hensel et al., 1989 |
| Factor | | |
| Thyroid Stimulating | Thyroid Hormone | Chatterjee et al., 1989 |
| Hormone α Gene | | |

TABLE 3
Other Promoter/Enhancer Elements

| Promoter/Enhancer | References |
|---|---|
| Immunoglobulin Heavy Chain | Hanerji et al., 1983; Gilles et al., |
| | 1983; Grossehedl and Baltimore, |
| | 1985; Atchinson and Perry, 1986, |
| | 1987; Imler et al., 1987; Weinberger |
| | et al., 1988; Kiledjian et al., 1988; |
| | Porton et al., 1990 |
| Immunoglobulin Light Chain | Queen and Baltimore, 1983; Picard |
| | and Schaffner, 1984 |
| T-Cell Receptor | Luria et al., 1987, Winoto and |
| | Baltimore, 1989; Redondo et al., |
| | 1990 |
| HLA DQ α and DQ β | Sullivan and Peterlin, 1987 |
| β-Interferon | Goodbourn et al., 1986; Fujita et al., |
| | 1987; Goodbourn and Maniatis, 1985 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRα | Sherman et al., 1989 |
| β-Actin | Kawamoto et al., 1988; Ng et al., |
| | 1989 |
| Muscle Creatine Kinase | Jaynes et al., 1988; Horlick and |
| | Benfield, 1989; Johnson et al., 1989a |
| Prealbumin (Transthyretin) | Costa et al., 1988 |
| Elastase I | Omitz et al., 1987 |
| Metallothionein | Karin et al., 1987; Culotta and |
| | Hamer, 1989 |
| Collagenase | Pinkert et al., 1987; Angel et al., |
| | 1987 |
| Albumin Gene | Pinkert et al., 1987, Tronche et al., |
| | 1989, 1990 |
| α-Fetoprotein | Godbout et al., 1988; Campere and |
| | Tilghman, 1989 |
| t-Globin | Bodine and Ley, 1987; Perez-Stable |
| | and Constantini, 1990 |
| β-Globin | Trudel and Constantini, 1987 |
| e-fos | Cohen et al., 1987 |
| c-HA-ras | Triesman, 1986; Deschamps et al., |
| | 1985 |
| Insulin | Edlund et al., 1985 |
| Neural Cell Adhesion | Hirsch et al., 1990 |
| Molecule (NCAM) | |
| α-1-antitrypsin | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., |
| | 1981; Sleigh and Lockett, 1985; Firak |
| | and Subramanian, 1986; Herr and |
| | Clarke, 1986; Imbra and Karin, 1986; |
| | Kadesch and Berg, 1986; Wang and |
| | Calame, 1986; Ondek et al., 1987; |
| | Kuhl el al., 1987 Schaffner et al., |
| | 1988 |
| Polyoma | Swartzendruber and Lehman, 1975; |
| | Vasseur et al., 1980; Katinka et al., |
| | 1980, 1981; Tyndell et al., 1981; |
| | Dandolo et al., 1983; deVilliers et al., |
| | 1984; Hen et al., 1986; Satake et al., |
| | 1988; Campbell and Villarreal, 1988 |
| Retroviruses | Kriegler and Botchan, 1982, 1983; |
| | Levinson et al., 1982; Kriegler et al., |
| | 1983, 1984a, b, 1988; Bosze et al., |
| | 1986; Miksicek et al., 1986; Celander |
| | and Haseltine, 1987; Thiesen et al., |
| | 1988; Celander et al., 1988; Chol et |
| | al., 1988; Reisman and Rotter, 1989 |
| Papilloma Virus | Campo et al., 1983; Lusky et al., |
| | 1983; Spandidos and Wilkie, 1983; |
| | Spalholz et al., 1985; Lusky and |
| | Botchan, 1986; Cripe et al., 1987; |
| | Gloss et al., 1987; Hirochika et al., |
| | 1987, Stephens and Hentschel, 1987; |
| | Glue et al., 1988 |
| Hepatitis B Virus | Bulla and Siddiqui, 1986; Jameel and |
| | Siddiqui, 1986; Shaul and Ben-Levy, |
| | 1987; Spandau and Lee, 1988 |
| Human Immunodeficiency Virus | Muesing et al., 1987; Hauber and |
| | Cullan, 1988; Jakobovits et al., 1988; |
| | Feng and Holland, 1988; Takebe et |
| | al., 1988; Rowen et al., 1988; |
| | Berkhout et al., 1989; Laspia et al., |
| | 1989; Sharp and Marciniak, 1989; |
| | Braddock et al., 1989 |
| Cytomegalovirus | Weber et al., 1984; Boshart et al., |
| | 1985; Foecking and Hofstetter, 1986 |
| Gibbon Ape Leukemia Virus | Holbrook et al., 1987; Quinn et al., |
| | 1989 |
| Dectin-1 | |
| Dectin-2 | |

The use of internal ribosome binding sites (IRES) elements are used to create multigene, or polycistronic, messages. IRES elements are able to bypass the ribosome scanning model of 5′-methylated cap-dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message. Another signal that may prove useful is a polyadenylation signal (hGH, BGH, SV40).

As discussed above, in certain embodiments of the invention, a cell may be identified and selected in vitro or in vivo by including a marker in the expression construct. Such markers confer an identifiable change to the cell permitting easy identification of cells containing the expression construct. Usually, the inclusion of a drug selection marker aids in cloning and in the selection of transformants, for example, genes that confer resistance to neomycin, puromycin, hygromycin, DHFR, GPT, zeocin, tetracycline and histidinol are useful selectable markers. Alternatively, enzymes such as herpes simplex virus thymidine kinase (tk) or chloramphenicol acetyltransferase (CAT) may be employed.

The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV 40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between elements is flexible, so that promoter function is preserved when elements are inverted or moved relative to one another. In the tk promoter, the spacing between elements can be increased to 50 bp apart before activity begins to decline. Depending on the promoter, it appears that individual elements can function either co-operatively or independently to activate transcription.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements On the other hand, a promoter must have one or more elements that direct initiation of RNA synthesis at a particular site and in a particular orientation, whereas enhancers lack these specificities. Aside from this operational distinction, enhancers and promoters are very similar entities.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way.

In any event, it will be understood that promoters are DNA elements which when positioned functionally upstream of a gene leads to the expression of that gene. Most transgene constructs of the present invention are functionally positioned downstream of a promoter element.

F. Pharmaceutical Compositions and Routes of Self Gene Delivery

In a preferred embodiment of the present invention, a method of treating a subject with a hyperproliferative disease in which self gene expression is increased or altered is contemplated. Hyperproliferative diseases that are most likely to be treated in the present invention are those that result from mutations in the self gene and the overexpression of self gene protein in the hyperproliferative cells. Examples of hyperproliferative diseases contemplated for treatment are lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon, breast and -bladder and any other hyperproliferative diseases that involve mutations and upregulation of self gene expression. An important aspect of this embodiment is the delivery of a self gene adenoviral vector to dendritic cells, for processing and presentation of self gene antigenic peptides to immune effector cells, thereby stimulating an anti-self gene response. In one embodiment, a self gene adenovirus concentration range of 100-300 PFU/cell transduces greater than 50% of the dendritic cells. The preferred mode of delivering the self gene construct in the present invention is by adenoviral vector.

In a preferred embodiment of the present invention, a method of treating a subject with a hyperproliferative disease in which p53 expression is upregulated is contemplated. Hyperproliferative diseases that are most likely to be treated in the present invention are those that result from mutations in the p53 gene and the overexpression of p53 protein in the hyperproliferative cells. Examples of hyperproliferative diseases contemplated for treatment are lung cancer, head and neck cancer, breast cancer, pancreatic cancer, prostate cancer, renal cancer, bone cancer, testicular cancer, cervical cancer, gastrointestinal cancer, lymphomas, pre-neoplastic lesions in the lung, colon, rectal, breast and -bladder and any other hyperproliferative diseases that involve mutations and upregulation of p53 expression. An important aspect of this embodiment is the delivery of a p53 adenoviral vector to dendritic cells, for processing and presentation of p53 antigenic peptides to immune effector cells, thereby stimulating an anti-p53 response. In one embodiment, a p53 adenovirus concentration range of 100-300 PFU/cell transduces greater than 50% of the dendritic cells. The preferred mode of delivering the p53 adenoviral vector construct in the present invention is by intradermal injection of dendritic cells. In certain embodiments, the injection site is pretreated with chemokines or cytokines to elicit dendritic cell migration and maturation to the site of intradermal injection. In further embodiments, administration of the self gene adenoviral vector to dendritic cells comprises multiple intradermal injections. For example, the treatment of certain cancer types may require at least 3 or more immunizations, every 2-4 weeks. Dendritic cell intradermal injection may further be performed local, regional, or distal to the site of tumor growth, as well as subqutaneous, intraperitoneal or injection into or near a draining lymph node. Identifying, isolating, and obtaining dendritic cells are described below, in section II.

In certain embodiments, the present invention also concerns formulations of one or more self gene adenovirus compositions for administration to a mammal, that transduces dendritic cells of the mammal. For the treatment of hyperproliferative disease in humans, it is contemplated that the adenovirus vector is replication-defective, comprising a self gene under the control of a promoter operable in eukaryotic cells (e.g., CMV IE, dectin-1, dectin-2). It will also be understood that, if desired, the self gene compositions disclosed herein may be administered in combination with other agents as well, such as, e.g., various pharmaceutically-active agents. As long as the composition comprises at least one self gene expression construct, there is virtually no limit to other components which may also be included, given that the additional agents do not cause a significant adverse effect upon contact with the dendritic cells.

Adjuvants are substances that non-specifically enhance or potentiate the immune response (e.g., CTLs) to an antigen, and would thus be considered useful in formulations of the present invention. For example, cholera toxin acts locally as a mucosal adjuvant for the induction of peptide-specific CTLs following intranasal immunization of dendritic cells with CTL epitope peptides (Porgador et al., 1997; Porgador et al., 1998). Several immunological adjuvants (e.g., MF59) specific for dendritic cells and their preparation have been described previously (Dupis et al., 1998; Allison, 1997; Allison, 1998). The use of such adjuvants in the present invention are considered. In another embodiment of the present invention, cytokines are used in combination with the delivery of the p53 expression construct. Cytokines are secreted, low-molecular weight proteins that regulate the intensity and duration of the immune response by exerting a variety of effects on lymphocytes and other immune cells. Several cytokines have been directly linked to influencing dendritic cell migration to lymphoid tissues (e.g., TNF-α), accelerating the maturation of dendritic cells into efficient antigen-presenting cells for T-lymphocytes (e.g., GM-CSF, IL-1 and IL-4) (Dupis et al., 1998; Allison, 1997; Allison, 1998; U.S. Pat. No. 5,849,589, specifically incorporated herein by reference in its entirety) and acting as immunoadjuvants (e.g., IL-12) (Gabrilovich et al., 1996). The use of these and other cytokines (e.g., FLT-3 ligand, CD 40) are considered in the present invention.

The formulation of pharmaceutically-acceptable excipients and carrier solutions are well-known to those of skill in the art, as is the development of suitable dosing and treatment regimens for using the particular compositions described herein in a variety of treatment regimens, including, e.g., intradermal, parenteral, intravenous, intramuscular, intranasal, and oral administration and formulation.

1. Injectable Compositions and Delivery

The preferred method of the self gene adenovirus expression construct delivery to dendritic cells in the present invention is via intradermal injection. However, the pharmaceutical compositions disclosed herein may alternatively be administered parenterally, intravenously, intramuscularly, or even intraperitoneally as described in U.S. Pat. No. 5,543,158; U.S. Pat. No. 5,641,515 and U.S. Pat. No. 5,399,363 (each specifically incorporated herein by reference in its entirety). Injection of self gene constructs and transduced dendritic cells may be delivered by syringe or any other method used for injection of a solution, as long as the expression construct or transduced cells can pass through the particular gauge of needle required for injection. A novel needeless injection system has recently been described (U.S. Pat. No. 5,846,233) having a nozzle defining an ampule chamber for holding the solution and an energy device for pushing the solution out of the nozzle to the site of delivery. A syringe system has also been described for use in gene therapy that permits multiple injections of predetermined quantities of a solution precisely at any depth (U.S. Pat. No. 5,846,225).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

2. Oral Compositions and Delivery

The pharmaceutical compositions disclosed herein may be delivered via oral administration to an animal, and as such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. No. 5,641,515; U.S. Pat. No. 5,580,579 and U.S. Pat. No. 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium-borate solution (Dobell's Solution). Alternatively, the active ingredient may be incorporated into an oral solution such as those containing sodium borate, glycerin and potassium bicarbonate, or dispersed in a dentifrice, including: gels, pastes, powders and slurries, or added in a therapeutically effective amount to a paste dentifrice that may include water, binders, abrasives, flavoring agents, foaming agents, and humectants, or alternatively fashioned into a tablet or solution form that may be placed under the tongue or otherwise dissolved in the mouth.

3. Additional Modes of Delivery

In addition to the methods of delivery described above, the following techniques are also contemplated as alternative methods of self gene delivery. Sonophoresis (i.e., ultrasound)has been used and described in U.S. Pat. No. 5,656,016 (specifically incorporated herein by reference in its entirety) as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. No. 5,770,219 and U.S. Pat. No. 5,783,208), rectal delivery (U.S. Pat. No. 5,811,128) and feedback controlled delivery (U.S. Pat. No. 5,697,899), each specifically incorporated herein by reference in its entirety.

G. Monitoring Immune Response

In one embodiment of the present invention, self gene adenovirus vectors are intradermally administered to dendritic cells. Subsequently, the dendritic cells express and present self gene antigens to immune effector cells, thereby stimulating an anti-self gene response. In another embodiment, the immune effector cells are cytotoxic T lymphocytes (CTLs). Thus, an important aspect of the invention is the ability to monitor immune responses, specifically CTLs.

1. CTL Assay

Cytotoxic T lymphocyte activity can be assessed in freshly isolated peripheral blood mononuclear cells (PBMC), in phytohaemaglutinin-stimulated IL-2 expanded cell lines established from PBMC (Bernard et al., 1998) or by T cells isolated from previously immunized subjects and restimulated for 6 days with DC infected with Adenovirus self gene using standard 6 h ⁵¹Cr release microtoxicity assays. Colonic T-cells have been tested for their ability to mediate both perforin and Fas ligand-dependent killing in redirected cytotoxicity assays (Simpson et al., 1998). The colon cytotoxic T lymphocytes displayed both Fas- and perforin-dependent killing. Recently, an in vitro dehydrogenase release assay has been developed that takes advantage of a new fluorescent amplification system (Page et al., 1998). This approach is sensitive, rapid, reproducible and may be used advantageously for mixed lymphocyte reaction (MLR). It may easily be further automated for large scale cytotoxicity testing using cell membrane integrity, and is thus considered in the present invention. In another fluorometric assay developed for detecting cell-mediated cytotoxicity, the fluorophore used is the non-toxic molecule alamar blue (Nociari et al., 1998). The alamarBlue is fluorescently quenched (i.e. low quantum yield) until mitochondrial reduction occurs, which then results in a dramatic increase in the alamarBlue fluorescence intensity (i.e. increase in the quantum yield). This assay is reported to be extremely sensitive, specific and requires a significantly lower number of effector cells than the standard ⁵¹Cr release assay.

2. Anti-CTL Antibodies

It is also contemplated in the present invention, that antibodies directed against specific CTL epitopes may be used to assay CTL immune responses. The culturing and activation of mononuclear leukocytes with a standard stimulus known to activate such cells has been described in U.S. Pat. No. 5,843,689 (specifically incorporated herein by reference in its entirety). After culturing, aliquots of the cells are incubated with fluorophore-conjugated monoclonal antibodies to antigenic determinants of a particular mononuclear subclass (e.g., CTLs). The incubated aliquots are analyzed on a flow cytofluorometer. It is contemplated that the use of CTL specific monoclonal antibodies and fluorophore-conjugated monoclonal antibodies (e.g., CD8+, FasL, CD4+) will be of particular use as assays in the present invention.

H. Ex Vivo Preparation of Dendritic Cells

In one embodiment of the present invention, a method for a p53-directed immune response in a subject is induced by: 1) obtaining dendritic cells from the subject, 2) infecting dendritic cells with an adenoviral vector comprising a p53 gene under the control of a promoter operable in eukaryotic cells and 3) the p53 adenovirus-infected dendritic cells are administered to the subject. It is contemplated that infected dendritic cells will present p53 antigens to immune effector cells and therefore stimulate an anti-p53 response in the subject. Thus, an important aspect of the present invention is to obtain dendritic cells from the subject or induce precursor cells (e.g., monocytes) to differentiate into dendritic cells -for infection with p53 adenoviral vectors for use in treatment of hyperproliferative disease.

It has been observed experimentally that patients with advanced stages of certain types of cancer have reduced function of dendritic cells (i.e. defective antigen presentation), but that these patients could give rise to functional dendritic cells through the in vitro growth and stimulation of stem cells (Gabrilovich et al., 1997). The stem cells were obtained from the cancer patients, stimulated to differentiate into dendritic cells by the addition of granulocyte/macrophage colony-stimulating factor and IL4, and observed to elicit much higher levels of CTL responses than mature dendritic cells obtained from the cancer patients (Gabrilovich et al., 1997). Thus, it is contemplated in the present invention that stem cell precursor stimulated dendritic cell differentiation is used as a method for ex vivo treatment of hyperproliferative disease.

A method of culturing and inducing the differentiation of monocytes into dendritic cells has been described in U.S. Pat. No. 5,849,589 (specifically incorporated herein by reference in its entirety). The method of monocyte differentiation into dendritic cells consists of a culture medium stimulated with GM-CSF, IL-4 and TNFα. An alternate method of isolating dendritic cells has been described by Cohen et al. (U.S. Pat. No. 5,643,786, specifically incorporated herein by reference in its entirety). This method involves elutriating peripheral blood samples in at least four flow rates from an elutriation rotor. Calcium ionophore is used to stimulate monocytes isolated during the process into dendritic cells and treatment for diseases involving re-introduction of the activated dendritic cells are also disclosed. It is also possible to prepare immortalized precursor cells that is considered useful in the present invention (U.S. Pat. No. 5,830,682; U.S. Pat. No. 5,811,297, each specifically incorporated herein by reference in its entirety). In another example, an immature dendritic cell line derived from p53 growth suppressor gene deficient animals are prepared (U.S. Pat. No. 5,648,219, specifically incorporated herein by reference in its entirety). The immature dendritic cell line may be induced to become an activated, immortalized dendritic cell line that will stimulate T-cell proliferation and is thus contemplated for use in the present invention. Methods and compositions for use of human dendritic cells to activate T-cells for immunotherapeutic responses against primary and metastatic prostate cancer have also been described (U.S. Pat. No. 5,788,963, specifically incorporated herein by reference in its entirety). After the exposure of the dendritic cells to prostate cancer antigen in vitro, the dendritic cells are administered to a prostate cancer patient to activate T-cell responses in vivo. An important embodiment of the invention described above (U.S. Pat. No. 5,788,963) is a method to extend the life span of the human dendritic cells by cryopreservation. This method may be of important utility in the present invention for long term storage of p53 adenoviral infected dendritic cells.

I. Pharmaceuticals and Methods of Treating Cancer

In a particular aspect, the present invention provides methods for the treatment of various hyperproliferative diseases. Treatment methods will involve treating an individual with an effective amount of a viral particle, as described above, containing a self gene of interest. An effective amount is described, generally, as that amount sufficient to detectably and repeatedly to ameliorate, reduce, minimize or limit the extent of the disease or its symptoms. More rigorous definitions may apply, including elimination, eradication or cure of disease.

To kill cells, inhibit cell growth, inhibit metastasis, decrease tumor or tissue size and otherwise reverse or reduce the malignant phenotype of tumor cells, using the methods and compositions of the present invention, one would generally contact a dendritic cell with the therapeutic expression construct. This may be combined with compositions comprising other agents effective in the treatment of hyperproliferative cells. These compositions would be provided in a combined amount effective to kill or inhibit proliferation of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent.

Alternatively, the dendritic cell therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the radio- or chemotherapeutic agent is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described dendritic cell therapy.

Aqueous compositions of the present invention comprise an effective amount of the compound, dissolved or dispersed in a pharmaceutically acceptable carrier or aqueous medium. Such compositions can also be referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

The treatments may include various “unit doses.” Unit dose is defined as containing a predetermined-quantity of the therapeutic composition calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time. Unit dose of the present invention may conveniently may be described in terms of plaque forming units (pfu) of the viral construct. Unit doses range from 10³, 10⁴, 10⁵, 10⁶, 10⁷, 10⁸, 10⁹, 10¹⁰, 10¹¹, 10¹², 10¹³ pfu and higher.

Preferably, patients will have adequate bone marrow function (defined as a peripheral absolute granulocyte count of >2,000/mm³ and a platelet count of 100,000/mm³), adequate liver function (bilirubin <1.5 mg/dl) and adequate renal function (creatinine <1.5 mg/dl).

1. Gene Therapy

One of the preferred embodiments of the present invention involves the use of viral vectors to deliver therapeutic genes to dendritic cells for the treatment of cancer. Cancer cells include cancers of the lung, brain, prostate, kidney, liver, ovary, breast, skin, stomach, esophagus, head and neck, testicles, colon, cervix, lymphatic system and blood. Of particular interest are non-small cell lung carcinomas including squamous cell carcinomas, adenocarcinomas and large cell undifferentiated carcinomas, tumor suppressors, antisense oncogenes, and inhibitors of apoptosis.

According to the present invention, one may treat the cancer by directly injection a tumor with the viral vector. Alternatively, the tumor may be infused or perfused with the vector using any suitable delivery vehicle. Local or regional administration, with respect to the tumor, also is contemplated. Finally, systemic administration may be performed. Continuous administration also may be applied where appropriate, for example, where a tumor is excised and the tumor bed is treated to eliminate residual, microscopic disease. Delivery via syringe or catherization is preferred. Such continuous perfusion may take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

For tumors of >4 cm, the volume to be administered will be about 4-10 ml (preferably 10 ml), while for tumors of <4 cm, a volume of about 1-3 ml will be used (preferably 3 ml). Multiple injections delivered as single dose comprise about 0.1 to about 0.5 ml volumes. The viral particles may advantageously be contacted by administering multiple injections to the tumor, spaced at approximately 1 cm intervals.

In certain embodiments, the tumor being treated may not, at least initially, be resectable. Treatments with therapeutic viral constructs may increase the resectability of the tumor due to shrinkage at the margins or by elimination of certain particularly invasive portions. Following treatments, resection may be possible. Additional viral treatments subsequent to resection will serve to eliminate microscopic residual disease at the tumor site.

A typical course of treatment, for a primary tumor or a post-excision tumor bed, will involve multiple doses. Typical primary tumor treatment involves a 6 dose application over a two-week period. The two-week regimen may be repeated one, two, three, four, five, six or more times. During a course of treatment, the need to complete the planned dosings may be re-evaluated.

2. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate or any analog or derivative variant thereof.

3. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

### J. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Animals

6-8 wk-old female BALB/c and CBA mice were purchased from Harlan Inc. (Indianapolis, Ind.) and were housed in specific pathogen-free units of the Division of Animal Care at Vanderbilt University Medical Center.

Reagents and Cell Lines

Tumor cells D459 were constructed by transfection of BALB/c 3T3 cells with EJ ras and a mutant human p53 expression vector. Details of this cell lines wore described elsewhere (Gabrilovich et al., 1996; Yanuck et al., 1993). MethA sarcoma cells were obtained from Dr. L. J. Old. This is a Ishida et al. transplantable 3-methylcholanthrene-induced sarcoma of BALB/c origin passaged as an ascitic tumor. P815 mouse mastocytoma cell lines transfected with mutant human p53 genes were also described elsewhere (Ciemik et al., 1995). These two cell lines contain p53 genes with two different mutations, one in codon 135 (P815-135) and the other one in codon 173 (P815-173).

Control adenovirus (Ad-c) was prepared by deletion of E1 region from adenovirus serotype 5. Adenovirus containing human wild-type 53 (Ad-p53) was obtained from Idtrogen Therapeutics Inc., Houston, Tex. Recombinant mouse GM-CSF and IL-4 were obtained from R&D Systems, Minneapolis, Minn.

FITC and PE labeled antibodies used in flow cytometry were purchased from Pharmigen (San Diego, Calif.): anti-CD11c (N418), anti-CD-86 (B7-2), anti-CD40 and anti-I-A^{d}. FITC- and PE-conjugated isotype matched IgG were used in controls. Anti-p53 antibodies were obtained from Dako Corporation, Capinteria, Calif. FITC 10 labeled anti-mouse Ig was obtained from Sigma, St. Louis, Mo.

Cell Preparation and Infection with Adenovirus

Bone marrow cells were prepared as described earlier (Gabrilovich et al., 1996). Briefly, bone marrow cells were obtained from the femurs and tibias of BALB/c mice. Mononuclear cells were placed in tussie flasks at a concentration 5×10⁵/ml in complete culture medium (CCM) (RPMI-1640, Gibco BRL, Gaithersburg, Md. with 100 IU/ml penicillin, 0.1 mg/mL streptomycin, 1×10⁵ M 2-mercaptoethanol and 10% fetal calf serum, HyClone, Logan, Utah) supplemented with rmGM-CSF at a final concentration 3 ng/ml and rmIL-4 at a final concentration of 5 ng/ml. After 3 days, half of the medium was removed after gentle swirling and replenished with an equivalent amount of fresh GM-CSF and IL-4 supplemented medium. 3-4 days later, clusters of DC were dislodged.

The purity of DC prepared in this fashion was greater than 60% at >95% viability.

Splenic DC were prepared as described (Gabrilovich et al., 1996). A single cell suspension was prepared by pressing the spleens through a wire mesh. Cells were then washed and incubated overnight in CCM. Non-adherent cells were layered onto a metrizamide (Nygaard, Oslo, Norway) gradient (14.5 g plus 100 ml RPMI 1640 medium) and centrifuged for 10 min at 600 g. Cells at the interface were washed once and resuspended in complete culture medium (CCM). DC's were identified by their distinctive morphology and by labeling with N418 (CD11c) antibody and had a purity >40% with >9S viability. T cells were isolated from lymph nodes using nylon wool columns as described elsewhere (Gabrilovich et al., 1996).

10⁶ DC obtained either from bone marrow or from spleen were infected with adenovirus at various multiplicities of infection (MOI) for 60 min in 1 ml of serun-free medium in 24-well plates. After that time, 1 ml of fresh medium supplemented with GM-CSF, IL-4 and 20% FCS was added. No IL-4 was added to splenic DC. Cells were incubated for another 24, 48, 72 or 120 h. After that time, cells were washed in PBS before use.

Tumor Induction and Immunization Procedures

For immunization, bone marrow derived DC were used. Two hundred thousand dendritic cells were injected either lv, lp or sc into BALB/c mice. Two hundred thousand D459 cells or 6×10³ MethA sarcoma cells were injected sc into the shaved backs of mice. These doses of tumor cells were chosen after preliminary studies showed that they resulted in tumor formation in 100% of the mice.

T Cell Proliferation Assay

DCs infected with Ad-p53 or Ad-c were irradiated (2000 cGy) and added in triplicate to 5×10⁴ T cells obtained from BALB/c mice immunized with Ad-p53 DC or, for a studies of allogeneic mixed leukocyte reaction (MLR), DCs were cultured with T cells obtained from CBA mice. After a 3 day incubation in 96 well U-bottomed plates, the cultures were pulsed with 1 μCi [³H]thymidine (Amersham, Arlington Heights, Ill.) for 8-12 h. [³H]Thymidine uptake was counted using a liquid scintillation counter.

Analysis of the p53 Protein Expression and Expression of Surface Molecules

The efficiency of DC transduction was tested based on the overexpression of human p53 protein by FACS analysis. Briefly, DC after infection with Ad-p53 or Ad-c were fixed for 30 min with 2% parafarmaldehyde, permeabilized for 60 min with 0.2% Tween 20 and stained with anti-p53 antibody. FITC conjugated anti-mouse Ig was used as a secondary antibody. Non-specific binding was measured using secondary antibody alone. Cells were analyzed using flow cytometer FACScalibur (Becton Dikinson, Mountain View, Calif.) with gates set around cluster of large cells. Expression of the surface molecules was studied on non-fixed, non-permeabilized DCs using monoclonal antibodies specific for B7-2, CD40, and lAd and analyzed by flow cytometry. Non-specific binding was measured using isotype matched mouse Ig.

CTL Assay

T cell cytotoxicity was measured in a standard 6 h ⁵¹Cr release assay. Briefly, 2×10⁶T cells isolated from immunized mice were restimulated for 6 days with 2×10⁵ splenic DC infected either with Ad-p53 or Ad-c in 24-well plates. Effector lymphocytes were incubated in duplicate with ⁵¹Cr labeled target cells. Supernatants were harvested with a Skatron Harvesting System (Skatron, Norway) and radioactivity was counted on a gamma counter. The percent specific lysis was calculated as 100×[(experimental release—spontaneous release)/maximum release—spontaneous release)].

### Example 2

### Determination of Effective AD-p53 Doses for Dendritic Cells Transduction

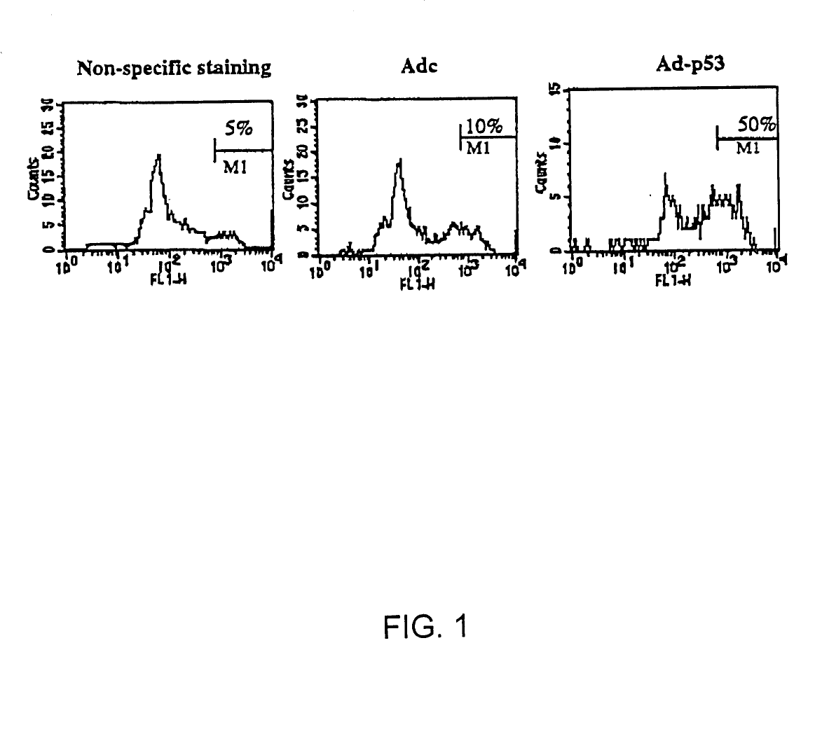

In preliminary studies, the most effective dose of Ad-p53 was determined. Ad-p53 and Ad-c at doses of 50-200 MOI did not significantly affect DC viability, which remained >95%. Higher doses of virus resulted in significant loss of viability (less than 50% at doses more than 500 MOI). The efficiency of transduction was estimated using intracellular staining with an anti-p53 antibody. The maximum level of p53 was detected at an Ad-p53 MOI of 100 pfu/cell. At this dose 40-45% DC were positive for p53 (FIG. 1). This dose of adenovirus was used in all subsequent studies. A 48 h incubation with Ad-p53 resulted in the highest levels of p53 protein, which slightly decreased 24 h later, and was undetectable by day 5. Thus, these preliminary studies demonstrated that Ad-p53 at a dose of 100 MOI was non-toxic for DC, and that Ad-p53-transduced DC expressed detectable levels of p53 protein. Infection of DC with adenovirus did not affect the ability of these cells to stimulate allogeneic T cells, and slightly increased expression of B7-2 and CD40 molecules on their surface.

### Example 3

### Determining the Efficacy of Immunization with AD-p53 Transduced DC in Eliciting an Anti-p53 Immune Response and How Many Immunizations are Required to Achieve the Effect

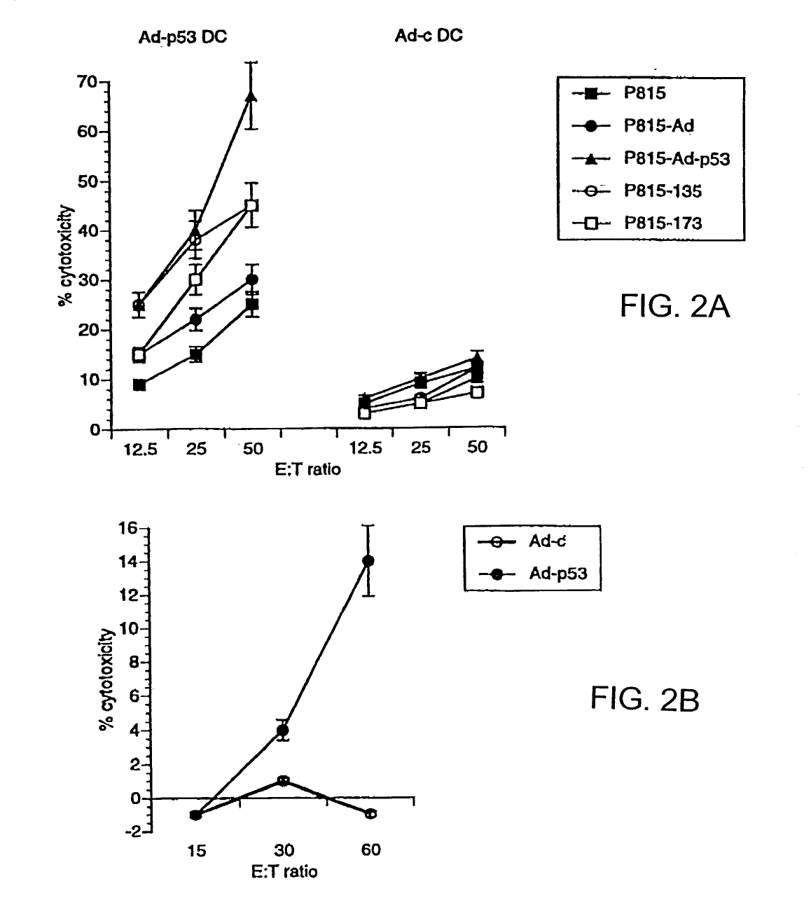

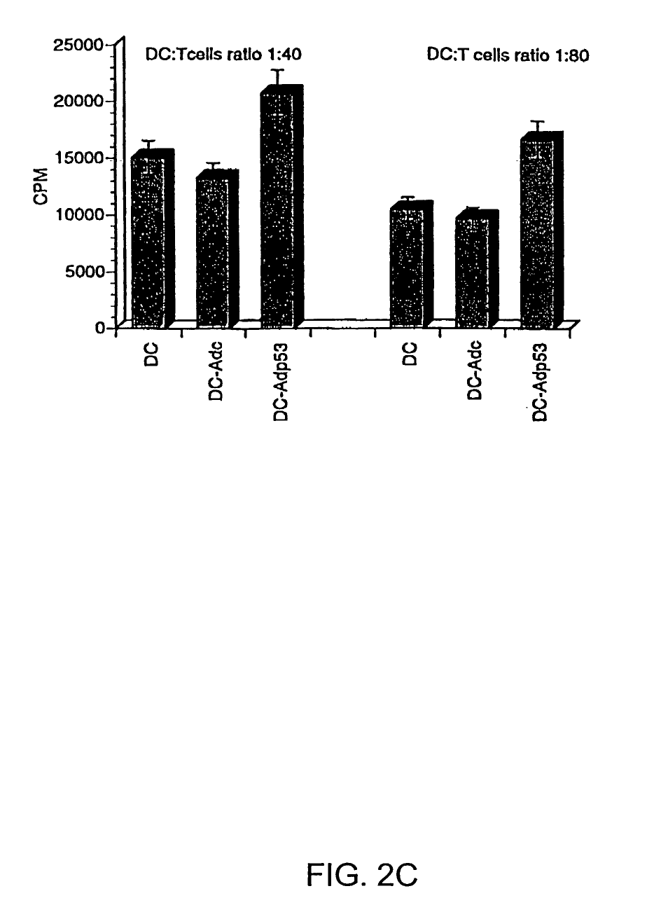

Mice were immunized with 2×10⁵ DC infected 48 h before with either Ad-p53 or Ad-c. Three routes of immunization were tested (sc, ip and iv) and immune responses were assayed using 5 different target tumors: P815 cells, P815 cells infected with control adenovirus (P815-Ad-c), P815 cells infected with Ad-p53 (P815-Ad-p53), P815-135 cells and P815-173 cells. Mice were immunized once or twice with a two wk interval. Ten to 14 days after the last immunization, T cells were isolated and restimulated with Ad-p53 infected splenic DC. No CTLs were detected after a single immunization using any of the tested routes of immunization. However, two immunization resulted in significant CTL responses (FIG. 2A). The highest response was observed against Ad-p53 infected P815 cells, however significant responses was also seen using P815-135 and P815-173 as targets. It is important to note the very low CTL responses detected against P815 cells infected with the control adenovirus. In three studies performed no differences in the level of anti-p53 specific CTL responses were found between the different routes of immunization

### EXample 4

### Immunization with AD-p53 DC Results in CTL Responses Against Mutant Murine p53

The studies described so far were performed using constructs and tumor cell lines expressing human p53. Since there is a high homology between human and murine p53, the inventors contemplated whether immunization with Ad-p53 DC also would result in CTL responses against overexpressed mutant murine p53. For these studies, the murine MethA sarcoma tumor was used, a carcinogen-induced tumor bearing different point mutations in each allele of its endogenous p53 genes. Tumor cells were preincubated for 3 days with 50 U/ml recombinant murine IFNγ and then used as a target in CTL assay. Low but clearly significant CTL responses specific for MethA were detected in mice immunized with Ad-p53 DC (FIG. 2B). Also tested was whether Ad-p53 DC were able to stimulate T cell proliferation in this system. T cells were obtained from immune mice (two immunizations with Ad-p53 DC) and were cultured with either uninfected DC (background level), or DC infected with Ad-c with Ad-p53. DC infected with Ad-p53, but not those infected with Ad-c were able to stimulate T cell proliferation significantly higher than background levels (FIG. 2C).

### Example 5

### CTL and T-cell Induced Immune Responses Provide Tumor Protection

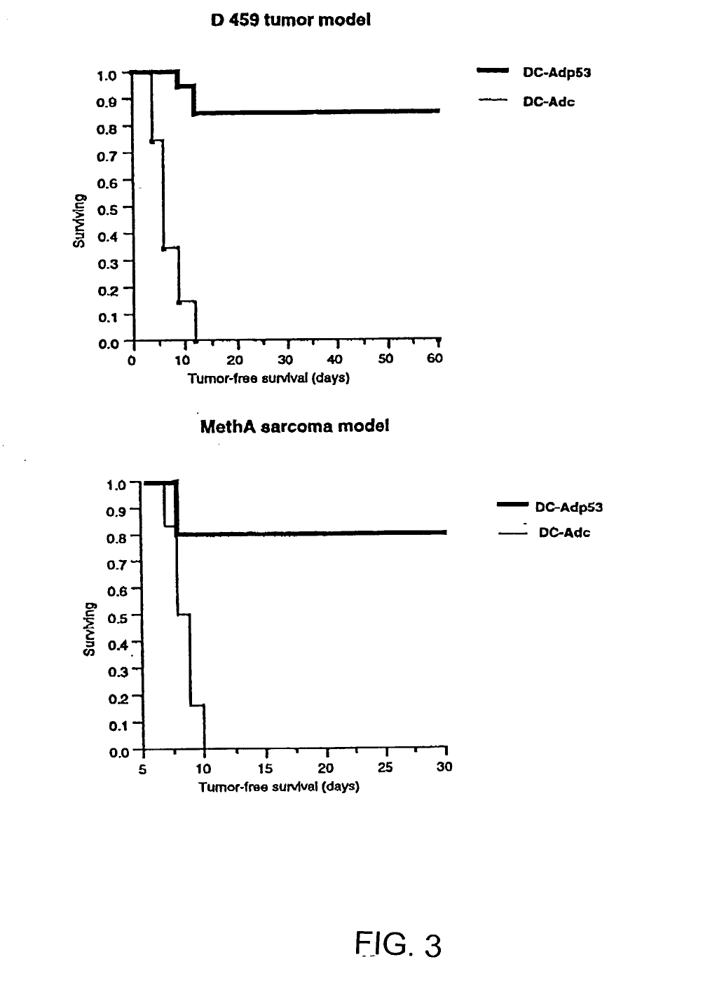

Mice were immunized twice iv with Ad-p53 and Ad-c infected DC. 10 days after the second immunization they were challenged with either D459 tumor, bearing a mutant human p53 gene, or with MethA sarcoma cells, expressing mutant murine p53. Doses of tumor cells were selected which resulted in tumor formation in 100% of non-immune control mice. After immunization with Ad-p53 DC, 17 out 20 (85%) immunized mice were completely protected against D459 tumor and 8 out 11 mice (72.7%) were protected against MethA sarcoma (FIG. 3).

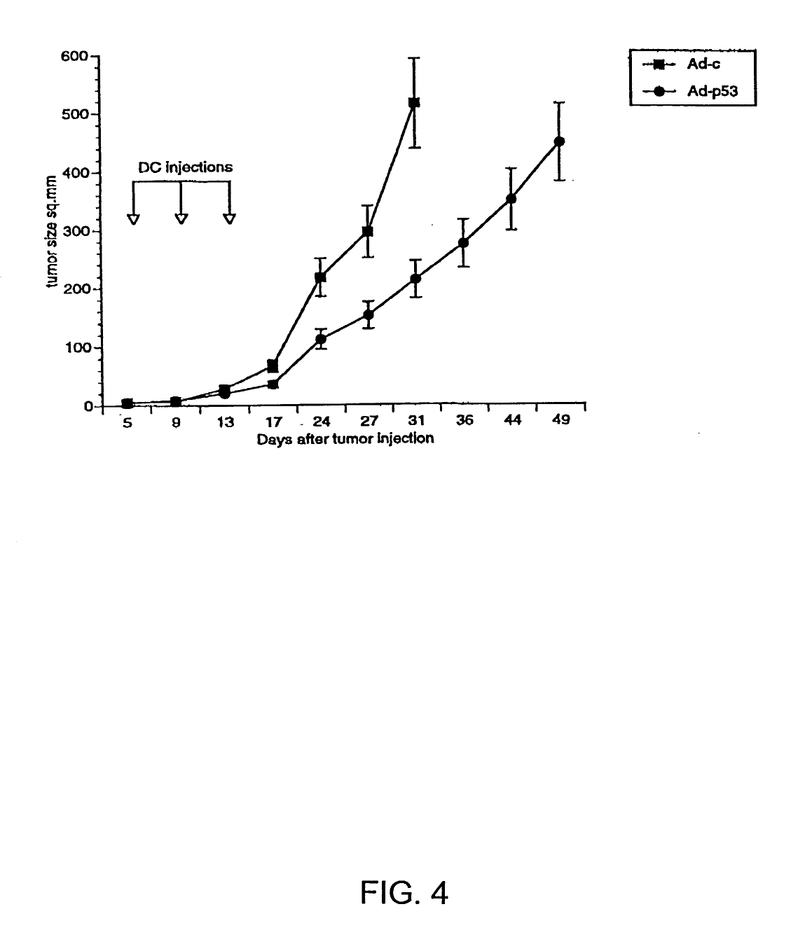

The inventors then investigated the effect of treatment of established poorly immunogeneic tumors with repeated injections with Ad-p53 infected DC. 2×10⁵ D459 were inoculated sc. When tumors became palpable, treatment with Ad-p53 DC was initiated. Mice were immunized three times and tumor growth was observed for 7 wk. Treatment with Adp53 infected DC significantly slowed down the tumor growth (FIG. 4). Mice in this group were sacrificed due to do bulky tumor more than two wk later than mice in the control group.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following references, to the extent that they provide exemplary procedural or other details supplementary to those set forth herein, are specifically incorporated herein by reference.

U.S. Pat. No. 4,430,434, issued Feb. 7, 1984

U.S. Pat. No. 4,452,901, issued Jun. 5, 1984.

U.S. Pat. No. 4,727,028, issued Feb. 23, 1988

U.S. Pat. No. 4,960,704, issued Oct. 2, 1990.

U.S. Pat. No. 5,399,363, issued Mar. 21, 1995.

U.S. Pat. No. 5,466,468, issued Nov. 14, 1995.

U.S. Pat. No. 5,543,158, issued Aug. 6, 1996.

U.S. Pat. No. 5,580,579, issued Dec. 3, 1996.

U.S. Pat. No. 5,602,306, issued Feb. 11, 1997

U.S. Pat. No. 5,633,016, issued May 27, 1997.

U.S. Pat. No. 5,639,940, issued Jun. 17, 1997.

U.S. Pat. No. 5,641,515, issued Jun. 24, 1997.

U.S. Pat. No. 5,643,786, issued Jul. 1, 1997.

U.S. Pat. No. 5,648,219, issued Jul. 15, 1997.

U.S. Pat. No. 5,656,016, issued Aug. 12, 1997.

U.S. Pat. No. 5,658,583, issued Aug. 19, 1997.

U.S. Pat. No. 5,697,899, issued Dec. 16, 1997.

U.S. Pat. No. 5,707,644, issued Jan. 13, 1998.

U.S. Pat. No. 5,718,914, issued Feb. 17, 1998.

U.S. Pat. No. 5,720,936, issued Feb. 24, 1998.

U.S. Pat. No. 5,725,871, issued Mar. 10, 1998.

U.S. Pat. No. 5,739,169, issued Apr. 14, 1998.

U.S. Pat. No. 5,747,469, issued May 5, 1998.

U.S. Pat. No. 5,756,353, issued May 26, 1998.

U.S. Pat. No. 5,770,219, issued Jun. 23, 1998.

U.S. Pat. No. 5,780,045, issued Jul. 14, 1998.

U.S. Pat. No. 5,783,208, issued Jul. 21, 1998.

U.S. Pat. No. 5,788,963, issued Aug. 4, 1998.

U.S. Pat. No. 5,789,655, issued Aug. 4, 1998.

U.S. Pat. No. 5,792,451, issued Aug. 11, 1998.

U.S. Pat. No. 5,797,898, issued Aug. 25, 1998.

U.S. Pat. No. 5,798,339, issued Aug. 25, 1998.

U.S. Pat. No. 5,801,005, issued Sep. 1, 1998.

U.S. Pat. No. 5,804,212, issued Sep. 8, 1998.

U.S. Pat. No. 5,811,128, issued Sep. 22, 1998.

U.S. Pat. No. 5,811,297, issued Sep. 22, 1998.

U.S. Pat. No. 5,814,599, issued Sep. 29, 1998.

U.S. Pat. No. 5,824,311, issued Oct. 20, 1998.

U.S. Pat. No. 5,824,346, issued Oct. 20, 1998.

U.S. Pat. No. 5,827,530, issued Oct. 27, 1998.

U.S. Pat. No. 5,830,682, issued Nov. 3, 1998.

U.S. Pat. No. 5,830,880, issued Nov. 3, 1998.

U.S. Pat. No. 5,836,935, issued Nov. 17, 1998.

U.S. Pat. No. 5,843,689, issued Dec. 1, 1998.

U.S. Pat. No. 5,846,225, issued Dec. 8, 1998.

U.S. Pat. No. 5,846,233, issued Dec. 8, 1998.

U.S. Pat. No. 5,846,928, issued Dec. 8, 1998.

U.S. Pat. No. 5,849,589, issued Dec. 15, 1998.

EPO 0273085

WPO 94/11514

Allison, “Immunological adjuvants and their modes of action,” Arch. Immunol. Ther. Exp. (Warsz), 45(203):141-147, 1997.

Allison, “The mode of action of immunological adjuvants,” Dev. Biol. Stand., 92:3-11, 1998.

Alt, Kellems, Bertino and Schimke, J. Biol. Chem., 253:1357, 1978.

Arthur et al., “A comparison of gene transfer methods in human dendritic cells,” Cancer Gene Ther., 4(1):17-25, 1997.

Austin-Ward, Villaseca, “Gene therapy and its applications,” Rev. Med. Chil., 126(7):838-45, 1998.

Baichwal et al., “Vectors for gene transfer derived from animal DNA viruses: Transient and stable expression of transferred genes,” In: Gene transfer, Kucherlapati R, ed., New York: Plenum Press, pp. 117-148, 1986.

Bernard et al., “HIV-specific cytotoxic T-lymphyocyte activity in immunologically normal HIV-infected persons,” AIDS, 12(16):2125-2139, 1998.

Bertholet et al., “Cytotoxic T lymphocyte responses to wild-type and mutant mouse p53 peptides,” Eur. J. Immunol., 27(3):798-801, 1997.

Bourlais et al., “Opthalmic drug delivery systems-recent advances.” Prog. Retin. Eye Res., 17:33-58, 1998.

Brizel, “Future directions in toxicity prevention,” Semin. Radiat. Oncol., 8(4 Suppl. 1):17-20, 1998.

Bukowski, Rayman, Uzzo, Bloom, Sandstrom, Peereboom, Olencki, Budd, McLain, Elson, Novick, Finke, “Signal transduiction abnormalities in T lymphocytes from patients with advanced renal carcinoma: clinical relevance and effects of cytokine therapy,” Clin. Cancer Res., 4(10):2337-47, 1998.

Caldovic and Hackett Jr., “Development of position-independent expression vectors and their transfer into transgenic fish,” Mol. Mar. Biol. Biotechnol., 4(1):51-61, 1995.

Caley, Betts, Davis, Swanstrom, Frelinger, Johnston, “Venezuelan equine encephalitis virus vectors expressing HIV-1 proteins: vector design strategies for improved vaccine efficacy,” Vaccine 17(23-24):3124-35, 1999.

Carver, Dalrymple, Wright, Cottom, Reeves, Gibson, Keenan, Barrass, Scott, Colman, et al., “Transgenic livestock as bioreactors: stable expression of human alpha-1-antitrypsin by a flock of sheep,” Biotechnology N.Y., 1 (11): 1263-1270, 1993.

Celluzzi and Falo, “Epidermal dendritic cells induce potent antigen-specific CTL-mediated immunity,“J. Invest. Dermatol., 108(5):716-720,1997.

Chalfie et al., Science, 263:802-805,1994.

Chang et al., “Foreign gene delivery and expression in hepatocytes using a hepatitis B virus vector,” Hepatology, 14:134A, 1991.

Chen and Okayama, “High-efficiency transfection of mammalian cells by plasmid DNA,” Mol. Cell Biol., 7:2745-2752, 1987.

Christodoulides, Brooks, Rattue, Heckels, “Immunization with recombinant class 1 outer-membrance protein from Neisseria meningitidis: influence of liposomes and adjuvants on antibody avidity, recognition of native protein and the induction of a bactericidal immune response against meningococci,” Microbiology, 144(Pt 11):3027-37, 1998.

Ciernik, Berzofsky, Carbone, “Induction of cytotoxic T lymphocytes and antitumor immunity with DNA vaccines expressing single T cell epitopes,” J. Immunol., 156(7):2369-75, 1996.

Clark, Voulgaropoulou, Fraley, Johnson, “Cell lines for the production of recombinant adeno-associated virus,” Human Gene Therapy, 6:1329-1341, 1995.

Coffin, “Retroviridae and their replication,” In: Virology, Fields et al. (eds.), New York: Raven Press, pp. 1437-1500, 1990.

Colberre-Garapin et al., J. Mol. Biol., 150:1, 1981.

Cotten, Wagner, Zatloukal, Phillips, Curiel, “High efficiency receptor-mediated delivery of small and large (48 kilobase) gene constructs using the endosome disruption activity of defective or inactivated adenovirus particles,” Proc. Natl. Acad. Sci. USA, 89:6094-6098, 1992.

Couch et al., “Immunization with types 4 and 7 adenovirus by selective infection of the intestinal tract,” Am. Rev. Resp. Dis., 88:394-403, 1963.

Coupar et al., “A general method for the construction of recombinant vaccinia virus expressing multiple foreign genes,” Gene, 68:1-10, 1988.

Cozzi, Tucker, Langford, Pino-Chavez, Wright, O'Connell, Young, Lancaster, McLanghlin, Hunt, Bordin, White, “Characterization of pigs transgenic for human decay-accelerating factor,” Transplantation, 64(10):1383-1392, 1997.

Curiel, “Gene transfer mediated by adenovirus-polylysine DNA complexes,” In: Viruses in Human Gene Therapy, J.-M. H. Vos (Ed.), Carolina Academic Press, Durham, N.C., pp. 179-212, 1994.

Curran, “Radiation-induced toxicities: the role of radioprotectants,” Semin. Radial. Oncol., 8(4 Suppl. 1):2-4, 1998.

Davidson, Musk, Wood, Morey, Ilton, Yu, Drury, Shilkin, Robinson, “Intralesional cytokine therapy in cancer: a pilot study of GM-CSF infusion in mesothelioma,” J. Immunother., 21(5):389-98, 1998.

DeLeo, “p53-based immunotherapy of cancer,” Crit. Rev. Immunol., 18(1-2):29-35, 1998.

Dupuis et al., “Dendritic cells internalize vaccine adjuvant after intramuscular injection,” Cell Immunol., 186(1):18-27, 1998.

Ebert, Selgrath, DiTulio, Denman, Smith, Memon, Schindler, Monastersky, Vitale, Gordon, “Transgenic production of a variant of human tissue-type plasminogen activator in goat milk: generation of transgenic goats and analysis of expression,” Biotechnology N.Y., 9(9):83 5-838, 1991.

el-Kareh, Secomb, “Theoretical models for drug delivery in solid tumors,” Crit. Rev. Biomed. Eng., 25(6):503-71, 1997.

Erlandsson, “Molecular genetics of renal cell carcinoma,” Cancer Genet. Cytogenet, 104(1):1-18, 1998.

Erlandsson, “Molecular genetics of renal cell carcinoma,” Cancer Genet. Cytogenet., 104(1):1-18, 1998.

Ferkol et al., FASEB J., 7:1081-1091, 1993.

Finlay et al., “Activating mutations for transformation by p53 produce a gene product that forms an hsc70-p53 complex with an altered half-life,” Mol. Cell. Biol., 8:531-539, 1988.

Flotte, Afione, Conrad, McGrath, Solow, Oka, Zeitlin, Guggino, and Carter, “Stable in vivo expression of the cystic fibrosis transmembrane conductance regulator with an adeno-associated virus vector,” Proc. Natl. Acad. Sci. USA, 90:10613-10617, 1993.

Flotte, Barraza-Ortiz, Solow, Afione, Carter, and Guggino, “An improved system for packaging recombinant adeno-associated virus vectors capable of in vivo transduction,” Gene Therapy, 2:29-37, 1995.

Flotte, Solow, Owens, Afione, Zeitlin, and Carter, “Gene expression from adeno associated virus vector in airway epithelial cells,” Am. J. Respir. Cell Mol. Biol., 7:349-356, 1992.

Fraley et al., “Entrapment of a bacterial plasmid in phospholipid vesicles: Potential for gene transfer,” Proc. Natl. Acad. Sci. USA, 76:3348-3352, 1979.

Franz, Mueller, Haartong, Frey, Katus, “Transgenic animal models: new avenues in cardiovascular physiology,” J. Mol. Med., 75(2):115-119, 1997.

Friedmann, “Progress toward human gene therapy,” Science, 244:1275-1281, 1989.

Fulci, Ishii, Van Meir, “p53 and brain tumors: from gene mutations to gene therapy,” Brain Pathol., 8(4):599-613, 1998.

Gabrilovich et al., “IL-12 and mutant P53 peptide-pulsed dendritic cells for the specific immunotherapy of cancer,” J. Immunother. Emphasis Tumor Immunol., 19(6):414-418, 1996.

Gabrilovich, Ciemik, Carbone, “Dendritic cells in antitumor immune responses. I. Defective antigen presentation in tumor-bearing hosts,” Cell Immunol., 170(1):101-10, 1996.

Gabrilovich et al., “Dendritic cells in antitumor immune responses. II. Dendritic cells grown from bone marrow precursors, but not mature DC from tumor-bearing mice, are effective antigen carriers in the therapy of established tumors,” Cell Immunol, 170(1):111-119, 1996.

Gabrilovich et al., “IL-12 and mutant P53 peptide-pulsed dendritic cells for the specific immunotherapy of cancer,” J. Immunother. Emphasis Tumor Immunol., 19(6):414-418, 1996.

Gabrilovich et al., “Decreased antigen presentation by dendritic cells in patients with breast cancer,” Clin. Cancer Res., 3(3):483-490, 1997.

Gertig and Hunter, “Genes and environment in the etiology of colorectal cancer,” Semin. Cancer Biol., 8(4):285-298, 1997.

Ghosh-Choudhury et al., “Protein IX, a minor component of the human adenovirus capsid, is essential for the packaging of full-length genomes,” EMBO J, 6:1733-1739, 1987.

Ghosh and Bachhawat, “Targeting of liposomes to hepatocytes,” In. Liver Diseases, Targeted Diagnosis and Therapy Using Specific Receptors and Ligands,” Wu G. and C. Wu ed. New York: Marcel Dekker, pp. 87-104, 1991.

Gomez-Foix et al., “Adenovirus-mediated transfer of the muscle glycogen phosphorylase gene into hepatocytes confers altered regulation of glycogen,” J. Biol. Chem., 267:25129-25134, 1992.

Gopal., “Gene transfer method for transient gene expression, stable transfection, and cotransfection of suspension cell cultures,” Mol. Cell Biol., 5:1188-1190, 1985.

Graham and Prevec, “Adenovirus-based expression vectors and recombinant vaccines,” Biotechnology, 20:363-390, 1992.

Graham and Prevec, “Manipulation of adenovirus vector,” In: Methods in Molecular Biology: Gene Transfer and Expression Protocol, E. J. Murray (ed.), Clifton, N.J.: Humana Press, 7:109-128, 1991.

Graham and van der Eb, “A new technique for the assay of infectivity of human adenovirus 5 DNA”, Virology, 52:456-467, 1973.

Graham et al., “Characteristics of a human cell line transformed by DNA from human adenovirus type 5”, J. Gen. Virol., 36:59-72, 1977.

Graham et al., “Characteristics of a human cell line transformed by DNA from human adenovirus type 5”, J. Gen. Virol., 36:59-72, 1977.

Grunhaus and Horwitz, “Adenovirus as cloning vector,” Seminar in Virology, 3:237-252, 1992.

Hanibuchi, Yano, Nishioka, Yanagawa, Kawano, Sone, “Therapeutic efficacy of mouse-human chimeric antiganglioside GM2 monoclonal antibody against multiple organ micrometastases of human lung cancer in NK cell-depleted SCID mice,” Int. J. Cancer, 78(4):480-5, 1998.

Harland and Weintraub, “Translation of mammalian mRNA injected into Xenopus oocytes is specifically inhibited by antisense RNA,” J. Cell Biol., 101:1094-1099, 1985.

Hartmann et al., “High frequency of p53 gene mutations in primary breast cancers in Japanese women, a low-incidence population,” Br. J. Cancer, 73(8):896-901, 1996.

Hartmann et al., “Overexpression and mutations of p53 in metastatic malignant melanomas,” Int. J. Cancer, 67(3):313-317, 1996.

Haskell and Bowen, “Efficient production of transgenic cattle by retroviral infection of early embryos,” Mol. Reprod. Dev., 40(3):386-390, 1995.

Hellstrand, Hermodsson, Naredi, Mellqvist, Brune, “Histamine and cytokine therapy,” Acta. Oncol., 37(4):347-53, 1998.

Hermonat and Muzyczka, “Use of adeno-associated virus as a mammalian DNA cloning vector; transduction of neomycin resistance into mammalian tissue culture cells,” Proc. Natl. Acad. Sci. USA, 81:6466-6470, 1984.

Hersdorffer et al., “Efficient gene transfer in live mice using a unique retroviral packaging line,” DNA Cell Biol., 9:713-723, 1990.

Herz and Gerard, “Adenovirus-mediated transfer of low density lipoprotein receptor gene acutely accelerates cholesterol clearance in normal mice,” Proc. Natl. Acad Sci. USA 90:2812-2816, 1993.

Ho, Lau, Leung, Johnson, “Internal radiation therapy for patients with primary or metastatic hepatic cancer: a review,” Cancer, 83(9):1894-907, 1998.

Horwich et al. “Synthesis of hepadenovirus particles that contain replication-defective duck hepatitis B virus genomes in cultured HuH7 cells,” J. Virol., 64:642-650, 1990.

Houbiers et al., “In vitro induction of human cytotoxic T lymphocyte responses against peptides of mutant and wild-type p53,” Eur. J. Immunol., 23(9):2072-2077, 1993.

Hui, Hashimoto, “Pathways for potentiation of immunogenicity during adjuvant-assisted immunizations with Plasmodium falciparum major merozoite surface protein 1,” Infect. Immun., 66(11):5329-36, 1998.

Hurpin et al., “The mode of presentation and route of administration are critical for the induction of immune responses to p53 and antitumor immunity,” Vaccine, 16(2-3):208-215, 1998.

Johnson and Hamdy, “Apoptosis regulating genes in prostate cancer (review),” Oncol. Rep., 5(3):553-557, 1998.

Johnson, Hamdy, “Apoptosis regulating genes in prostate cancer (review),” Oncol. Rep., 5(3):553-7, 1998.

Jones and Shenk, “Isolation of deletion and substitution mutants of adenovirus type 5,” Cell, 13:181-188, 1978.

Kaneda et al., “Increased expression of DNA cointroduced with nuclear protein in adult rat liver,” Science, 243:375-378, 1989.

Kang, Cho, Kole, “Up-regulation of luciferase gene expression with antisense oligonucleotides: implications and applications in functional assay development,” Biochemistry, 37(18):6235-6239, 1998.

Kaptitt, Leone, Samulski, Siao, Pfaff, O'Malley, During, “Long-term gene expression and phenotypic correction suing adeno-associated virus vectors in the mammalian brain,” Nature Genetics, 8:148-154, 1994.

Karlsson et al., EMBO J., 5:2377-2385, 1986.

Kato et al., “Expression of hepatitis β virus surface antigen in adult rat liver,” J. Biol. Chem., 266:3361-3364, 1991.

Kaufman, “Selection and Coamplification of Heterologous Genes in Mammalian Cells,” Methods in Enzymology, 185:537-566, 1990.

Kelleher and Vos, “Long-term episomal gene delivery in human lymphoid cells using human and avian adenoviral-assisted transfection,” Biotechniques, 17(6):1110-1117, 1994.

Klein et al., “High-velocity microprojectiles for delivering nucleic acids into living cells,” Nature, 327:70-73, 1987.

Kolmel, “Cytology of neoplastic meningosis,” J. Neurooncol., 38(2-3):121-125, 1998.

Kotin, Siniscalco, Samulski, Zhu, Hunter, McLaughlin, Muzyczka, Bems, “Site-specific integration by adeno-associated virus,” Proc. Natl. Acad. Sci. USA, 87:2211-2215, 1990.

Kovach, Hartmann, Blaszyk, Cunningham, Schaid, and Sommer, “Mutation detection by highly sensitive methods indicates that p53 gene mutations in breast cancer can have important prognostic value,” Proc. Natl. Acad. Sci. USA, 93:1093-1096, 1996.

LaFace, Hermonat, Wakeland, Peck, “Gene transfer into hematopoietic progenitor cells mediated by an adeno-associated virus vector,” Virology, 162:483-486, 1988.

Laughlin, Cardellichio, Coon, “Latent Infection of KB Cells with Adeno-Associated Virus Type 2,” J. Virol., 60:515-524, 1986.

Le Gal La Salle et al., “An adenovirus vector for gene transfer into neurons and glia in the brain,” Science, 259:988-990, 1993.

Lebkowski, McNally, Okarma, and Lerch, “Adeno-associated virus: a vector system for efficient introduction and integration of DNA into a variety of mammalian cell types,” Mol. Cell. Biol., *:3988-3996, 1988.

Leitner, Ying, Driver, Dubensky, Restifo, “Enhancement of tumor-specific immune response with plasmid DNA replicon vectors,” Cancer Res 60(1):51-5, 2000.

Levine, A. J., Momand, J., and Finlay, C. A. “The p53 tumor suppresor gene,” Nature, 351:453-456, 1991.

Levrero et al., “Defective and nondefective adenovirus vectors for expressing foreign genes in vitro and in vivo,” Gene, 101:195-202, 1991.

Liebermann, Gregory, Hoffman, “AP-1 (Fos/Jun) transcription factors in hematopoietic differentiation and apoptosis,” Int. J. Oncol., 12(3):685-700, 1998.

Luo, Zhou, Cooper, Munshi, Boswell, Broxmeyer, Srivastava, “Adeno-associated virus 2 mediated transfer and functional expression of a gene encoding the human granulocyte-macrophage colony-stimulating factor,” Blood, 82 (Supp.): 1,303A, 1994.

Magi-Galluzzi, Murphy, Cangi, Loda, “Proliferation apoptosis and cell cycle regulation in prostatic carcinogenesis,” Anal. Quant. Cytol. Histol., 20(5):343-350, 1998.

Mangray and King, “Molecular pathobiology of pancreatic adenocarcinoma,” Front Biosci., 3:D1148-1160, 1998.

Mann et al., “Construction of a retrovirus packaging mutant and its use to produce helper-free defective retrovirus,” Cell, 33:153-159, 1983.

Markowitz et al., “A safe packaging line for gene transfer: Separating viral genes on two different plasmids,” J. Virol., 62:1120-1124, 1988.

Mathiowitz et al., “Biologically erodable microspheres as potential oral drug delivery systems.” Nature, 386:410-414, 1998.

Mayer, Future developments in the selectivity of anticancer agents: drug delivery and molecular target strategies,” Cancer Metastasis Rev., 17(2):211-8, 1998.

Mayordomo et al., “Therapy of murine tumors with p53 wild-type and mutant sequence peptide-based vaccines,” J. Exp. Med., 183(4):1357-1365, 1996.

McCarty, Christensen, Muzyczka, “Sequences Required for Coordinate Induction of Adeno-Associated Virus p19 and p40 Promoters by Rep Protein,” J. Virol., 65:2936-2945, 1991.

McLaughlin, Collis, Hermonat, Muzyczka, “Adeno-Associated Virus General Transduction Vectors: Analysis of Proviral Structures,” J. Virol., 62:1963-1973, 1988.

Mougin, Bernard, Lab, “Biology of papillomavirus II infections. Their role in the carcinogenesis of the cervix,” Ann. Biol. Clin. (Paris), 56(1):21-8, 1998.

Mumby and Walter, “Protein phosphatases and DNA tumor viruses: transformation through the back door?” Cell Regul., 2(8):589-598, 1991.

Muzyczka, “Use of Adeno-Associated Virus as a General Transduction Vector for Mammalian Cells,” Curr. Top. Microbiol. Immunol., 158:97-129, 1992.

Natoli, Costanzo, Guido, Moretti, Levrero, Apoptotic, non-apoptotic, and anti-apoptotic pathways of tumor necrosis factor signalling,” Biochem. Pharmacol., 56(8):915-920, 1998.

Nicolas and Rubinstein, “Retroviral vectors,” In: Vectors: A survey of molecular cloning vectors and their uses, Rodriguez and Denhardt (eds.), Stoneham: Butterworth, pp. 494-513, 1988.

Nicolau and Sene, “Liposome-mediated DNA transfer in eukaryotic cells,” Biochem. Biophys. Acta, 721:185-190, 1982.

Nijman et al., “p53, a potential target for tumor-directed T cells,” Immunol. Letters, 40:171-178, 1994.

Nociari et al., “A novel one-step, highly sensitive fluorometric assay to evaluate cell-mediated cytotoxicity,” J. Immunol. Methods, 213(2): 157-167, 1998.

Ochi, Hasuoka, Mizushima, Matsumura, Harada, “A case of small pancreatic cancer diagnosed by serial follow-up studies promptly by a positive K-ras point mutation in pure pancreatic juice,” Am. J. Gastroenterol., 93(8):1366-1368, 1998.

Ohara, “Radiotherapy: a significant treatment option in management of prostatic cancer,” Gan. To. Kagaku. Ryoho., 25(6):823-8, 1998.

Ohi, Dixit, Tillery, and Plonk, “Construction and replication of an adeno-associated virus expression vector that contains human λ-globin cDNA,” Gene, 89L:279-282, 1990.

Oldstone et al., “A common antiviral cytotoxic T-lymphocyte epitope for diverse major histocompatibility complex haplotypes: implications for vaccination,” Proc. Natl. Acad. Sci. USA, 89:2752-2755, 1992.

Ono, Hirose, Miyazaki, Yamamoto, Matsumoto,“Transgenic medaka fish bearing the mouse tyrosinase gene: expression and transmission of the transgene following electroporation of the orange-colored variant,” Pigment Cell Res., 10(3): 168-175, 1997.

Page et al., “A non isotopic method for the meausrement of cell membrane integrity,” Anticancer Res., 18(4A):2313-2316, 1998.

Paskind et al., “Dependence of moloney murine leukemia virus production on cell growth,” Virology, 67:242-248, 1975.

Pelletier and Sonenberg, Nature, 334:320-325, 1988.

Perales et al., Proc. Natl. Acad. Sci. USA, 91:4086-4090, 1994.

Pietras, Pegram, Finn, Maneval, Slamon, “Remission of human breast cancer xenografts on therapy with humanized monoclonal antibody to HER-2 receptor and DNA-reactive drugs,” Oncogene, 17(17):2235-49, 1998.

Pinto-Sietsma and Paul, “Transgenic rats as models for hypertension,” J. Hum. Hypertens., 11(9):577-581, 1997.

Porgador et al., “Intranasal immunization with cytotoxic T-lymphocyte epitope peptide and mucosal adjuvant cholera toxin: selective augmentation of peptide-presenting dendritic cells in nasal mucosa-associated lymphoid tissue,” Infect. Immun., 66(12):5876-5881, 1998.

Potter et al., “Enhancer-dependent expression of human k immunoglobulin genes introduced into mouse pre-B lymphocytes by electroporation,” Proc. Nat'l Acad. Sci. USA, 81:7161-7165, 1984.

Qin, Tao, Dergay, Moy, Fawell, Davis, Wilson, Barsoum, “Interferon-beta gene therapy inhibits tumor formation and causes regression of established tumors in immune-deficient mice,” Proc. Natl. Acad. Sci. USA, 95(24):1411-6, 1998.

Racher et al., Biotechnology Techniques, 9:169-174, 1995.

Ragot et al., “Efficient adenovirus-mediated transfer of a human minidystrophin gene to skelet al muscle of mdx mice,” Nature, 361:647-650, 1993.

Renan, “Cancer genes: current status, future prospects, and applicants in radiotherapy/oncology,” Radiother. Oncol., 19:197-218, 1990.

Rich et al., “Development and analysis of recombinant adenoviruses for gene therapy of cystic fibrosis,” Hum. Gene Ther., 4:461-476, 1993.

Ridgeway, “Mammalian expression vectors,” In: Rodriguez R L, Denhardt D T, ed. Vectors: A survey of molecular cloning vectors and their uses. Stoneham: Butterworth, pp. 467-492, 1988.

Rippe et al., “DNA-mediated gene transfer into adult rat hepatocytes in primary culture,” Mol. Cell Biol., 10:689-695, 1990.

Ropke et al., “Spontaneous human squamous cell carcinomas are killed by a human cytotoxic T lymphocyte clone recognizing a wild-type p53-derived peptide,” Proc. Natl. Acad. Sci. USA, 93:14704-14707, 1996.

Rosenfeld et al., “Adenovirus-mediated transfer of a recombinant a1-antitrypsin gene to the lung epithelium in vivo,” Science, 252:431-434, 1991.

Rosenfeld et al., “In vivo transfer of the human cystic fibrosis transmembrane conductance regulator gene to the airway epitheliurm,” Cell, 68:143-155, 1992.

Roux et al., “A versatile and potentially general approach to the targeting of specific cell types by retroviruses: Application to the infection of human cells by means of major histocompatibility complex class I and class II antigens by mouse ecotropic murine leukemia virus-derived viruses,” Proc. Natl. Acad. Sci. USA, 86:9079-9083, 1989.

Samulski, Chang, Shenk, “Helper-free stocks of recombinant adeno-associated viruses: Normal integration does not require viral gene expression,” J. Virol., 63:3822-3828, 1989.

Samulski, Zhu, Xiao, Brook, Housman, Epstein, Hunter, “Targeted integration of adeno-associated virus (AAV) into human chromosome 19,” EMBO J., 10:3941-3950, 1991.

Santerre, et al., Gene, 30:147, 1984

Saurwein-Teissl et al., “Whole virus influenza vaccine activates dendritic cells (DC) and stimulates cytokine production by peripheral blood mononuclear cells (PBMC) while subunit vaccines support T cell proliferation,” Clin. Exp. Immunol., 114(2):271-276, 1998.

Shelling and Smith, “Targeted integration of transfected and infected adeno-associated virus vectors containing the neomycin resistance gene,” Gene Therapy, 1:165-169, 1994.

Simpson et al., “Consequences of Fas-ligand and perforin expression by colon T cells in a mouse model of inflammatory bowel disease,” Gastroenterology, 115(4):849-855, 1998.

Soddu and Sacchi, “p53: prospects for cancer gene therapy,” Cytokines Cell Mol. Ther., 4(3):177-185, 1998.

Solyanik, Berezetskaya, Bulkiewicz, Kulik, “Different growth patterns of a cancer cell population as a function of its starting growth characteristics: analysis by mathematical modelling,” Cell Prolif., 28(5):263-278, 1995.

Sonderbye el al., “In vivo and in vitro modulation of immune stimulatory capacity of primary dendritic cells by adenovirus-mediated gene transduction,” Exp. Clin. Immunogenet., 15(2):100-111, 1998.

Steinman, “The dendritic cell system and its role in immunogenecity,” Annu. Rev. Immunol., 9:271-296, 1991.

Stokke, Smedshammer, Jonassen, Blomhoff, Skarstad, Steen, “Uncoupling of the order of the S and M phases: effects of staurosporine on human cell cycle kinases,” Cell Prolif., 30(5):197-218, 1997.

Stratford-Perricaudet and Perricaudet, “Gene transfer into animals: the promise of adenovirus,” In: Human Gene Transfer, Eds, O. Cohen-Haguenauer and M. Boiron, Editions John Libbey Eurotext, France, p. 51-61, 1991.

Stratford-Perricaudet et al., “Evaluation of the transfer and expression in mice of an enzyme-encoding gene using a human adenovirus vector,” Hum. Gene Ther., 1:241-256, 1990.

Temin, “Retrovirus vectors for gene transfer: Efficient integration into and expression of exogenous DNA in vertebrate cell genome,” In: Gene Transfer, Kucherlapati (ed.), New York, Plenum Press, pp. 149-188, 1986.

Theobald et al., “Targeting p53 as a general tumor antigen,” Proc. Natl. Acad. Sci. USA, 92:11993011997, 1995.

Top et al., “Immunization with live types 7 and 4 adenovirus vaccines. II. Antibody response and protective effect against acute respiratory disease due to adenovirus type 7,” J. Infect. Dis., 124:155-160, 1971.

Tratschin, Miller, Smith, Carter, “Adeno-associated virus vector for high-frequency integration, expression and rescue of genes in mammalian cells,” Mol. Cell. Biol., 5:32581-3260, 1985.

Tratschin, West, Sandbank, Carter, “A human parvovirus, adeno-associated virus, as a eucaryotic vector: transient expression and encapsidation of the procaryotic gene for chloramphenicol acetyltransferase,” Mol. Cell. Biol., 4:2072-2081, 1984.

Tur-Kaspa et al., “Use of electroporation to introduce biologically active foreign genes into primary rat hepatocytes,” Mol. Cell Biol., 6:716-718, 1986.

Van Cott, Lubon, Russell, Butler, Gwazdauskas,-Knight, Drohan, Velander, “Phenotypic and genotypic stability of multiple lines of transgenic pigs expressing recombinant human protein C,” Transgenic Res., 6(3):203-212, 1997.

Vogelstein and Kinzler, “p53 function and dysfunction,” Cell, 70(4):523-526, 1992.

Wagner et al., Science, 260:1510-1513, 1990.

Wahlstrom et al., Mol. Endrocrinol., 6:1013-1022, 1992.

Walsh, Nienhuis, Samulski, Brown, Miller, Young, Liu, “Phenotypic correction of Fanconi anemia in human hematopoietic cells with a recombinant adeno-associated virus vector,” Proc. Natl. Acad. Sci. USA, 89:7257-7261, 1994; J. Clin. Invest., 94:1440-1448, 1994.

Watanabe, M., Shirayoshi, Y., Koshimizu, U., Hashimoto, S., Yonehara, S., Eguchi, Y., Tsujimoto, Y. and Nakatsuji, N., “Gene transfection of mouse primordial germ cells in vitro and analysis of their survival and growth control,” Exp. Cell Res. 230:76-83, 1997.

Wei, Wei, Samulski, Barranger, “Expression of the human glucocerebrosidase and arylsulfatase A genes in murine and patient primary fibroblasts transduced by an adeno-associated virus vector,” Gene Therapy, 1:261-268, 1994.

Wong et al., “Appearance of β-lactamase activity in animal cells upon liposome mediated gene transfer,” Gene, 10:87-94, 1980.

Wu and Wu, “Evidence for targeted gene delivery to HepG2 hepatoma cells in vitro,” Biochemistry, 27:887-892, 1988.

Wu and Wu, “Receptor-mediated in vitro gene transfections by a soluble DNA carrier system,” J. Biol. Chem., 262:4429-4432, 1987.

Wu and Wu, Adv. Drug Delivery Rev., 12:159-167, 1993.

Yang et al., “In vivo and in vitro gene transfer to mammalian somatic cells by particle bombardment,” Proc. Nat'l Acad. Sci. USA, 87:9568-9572, 1990.

Yang, Chen, Trempe, “Characterization of cell lines that inducibly express the adeno-associated virus Rep proteins,” J. Virol., 68:4847-4856, 1994.

Yanuck, Carbone, Pendleton, Tsukui, Winter, Minna, Berzofsky, “A mutant p53 tumor suppressor protein is a target for peptide-induced CD8+cytotoxlc T-cells, Cancer Res., 53(14):3257-61, 1993.

Yeom, Fuhrmann, Ovitt, Brehm, Ohbo, Gross, Huibner, Scholer, “Germline regulatory element of Oct-4 specific for totipotent cycle of embryonal cells,” Development, 122:881-894, 1996.

Yoder, Kang, Zhou, Luo, Srivastava, “In vivo gene transfer in murine hematopoietic reconstituting stem cells mediated by the adeno-associated virus 2-based vectors,” Blood, 82 (Supp.):1:347A, 1994.

Zhou, Broxmyer, Cooper, Harrington, Srivastava, “Adeno-associated virus 2 mediated gene transfer in murine hematopoietic cells, Exp. Hematol. (NY), 21:928-933, 1993.

Zhou, Cooper, Kang, Ruggieri, Heimfeld, Srivastava, Broxmeyer, “Adeno-associated virus 2-mediated high efficiency gene transfer into immature and mature subsets of hematopoietic progenitor cells in human umbilical cord blood,” J. Exp. Med., 179:1867-1875, 1994.

What is claimed is:

1. A method for treating a subject with a hyperproliferative disease comprising the steps of:
(i) identifying a subject with a hyperproliferative disease characterized by alteration or increased expression of a self gene product in at least some of the hyperproliferative cells in said patient; and
(ii) intradermally administering to said subject an expression construct comprising a self gene under the control of a promoter operable in eukaryotic dendritic cells,
whereby said self gene product is expressed by dendritic cells and presented to immune effector cells, thereby stimulating an anti-self gene product response.

2. The method of claim 1, wherein said self-gene product is an oncogene.

3. The method of claim 2, wherein said oncogene is selected from the group consisting of tumor suppressors, tumor associated genes, growth factors, growth-factor receptors, signal transducers, hormones, cell cycle regulators, nuclear factors, transcription factors and apoptic factors.

4. The method of claim 3, wherein said tumor suppressor is selected from the group consisting of Rb, p53, p16, p19, p21, p73, DCC, APC, NF-1, NF-2, PTEN, FHIT, C-CAM, E-cadherin, MEN-I, MEN-II, ZAC1, VHL, FCC, MCC, PMS1, PMS2, MLH-1, MSH-2, DPC4, BRCA1, BRCA2 and WT-1.

5. The method of claim 3, wherein said growth-factor receptor is selected from the group consisting of FMS, ERBB/HER, ERBB-2/NEU/HER-2, ERBA, TGF-β receptor, PDGF receptor, MET, KIT and TRK.

6. The method of claim 3, wherein said signal transducer is selected from the group consisting of SRC, AB1, RAS, AKT/PKB, RSK-1, RSK-2, RSK-3, RSK-B, PRAD, LCK and ATM.

7. The method of claim 3, wherein said transcription factor or nuclear factor is selected from the group consisting of JUN, FOS, MYC, BRCA1, BRCA2, ERBA, ETS, EVII, MYB, HMGI-C, HMGI/LIM, SKI, VHL, WT1, CEBP-β, NFKB, IKB, GL1 and REL.

8. The method of claim 3, wherein said growth factor is selected from the group consisting of SIS, HST, INT-1/WT1 and INT-2.

9. The method of claim 3, wherein said apoptic factor is selected from the group consisting of Bax, Bak, Bim, Bik, Bid, Bad, Bcl-2, Harakiri and ICE proteases.

10. The method of claim 3, wherein said tumor associated gene is selected from the group consisting of CEA, mucin, MAGE and GAGE.

11. The method of claim 4, wherein said tumor suppressor product is p53.

12. The method of claim 1, wherein said expression construct is a viral vector.

13. The method of claim 12, wherein said viral vector is an adenoviral vector, a retroviral vector, a vaccinia viral vector, an adeno-associated viral vector, a polyoma viral vector, an alphavirus vector, or a herpesviral vector.

14. The method of claim 13, wherein said viral vector is an adenoviral vector.

15. The method of claim 14, wherein said adenoviral vector is replication-defective.

16. The method of claim 15, wherein the replication defect is a deletion in the E1 region of the virus.

17. The method of claim 16, wherein the deletion maps to the E1B region of the virus.

18. The method of claim 17, wherein the deletion encompasses the entire E1B region of the virus.

19. The method of claim 18, wherein the deletion encompasses the entire E1 region of the virus.

20. The method of claim 1, wherein said promoter is selected from the group consisting of CMV IE, human or murine dectin-1, human or murine dectin-2, human CD11c, mammalian F4/80 and human or murine MHC class II.

21. The method of claim 20, wherein said promoter is CMV IE.

22. The method of claim 1, wherein said expression vector further comprises a polyadenylation signal.

23. The method of claim 1, wherein said hyperproliferative disease is cancer.

24. The method of claim 23, wherein said cancer is selected from the group consisting of lung, head, neck, breast, pancreatic, prostate, renal, bone, testicular, cervical, gastrointestinal, lymphoma, brain, colon, skin and bladder.

25. The method of claim 1, wherein said hyperproliferative disease is selected from the group consisting of RA, IBD, OA, leiomyomas, adenomas, lipomas, hemangiomas, fibromas, melanomas, restenosis, pre-neoplastic lesions in the lung and psoriasis.

26. The method of claim 1, wherein said expression construct is administered via injection.

27. The method of claim 26, further comprising multiple injections.

28. The method of claim 26, wherein the injection is performed local to a hyperproliferative or tumor site.

29. The method of claim 26, wherein the injection is performed regional to a hyperproliferative or tumor site.

30. The method of claim 26, wherein the injection is performed distal to a hyperproliferative or tumor site.

31. The method of claim 1, wherein intradermal administration is via continuous infusion.

32. The method of claim 1, wherein said subject is a human.

33. The method of claim 1, wherein said immune effector cells are CTLs.

34. The method of claim 1, further comprising administering to said subject at least a first cytokine.

35. The method of claim 34, further comprising administering to said subject a second cytokine, different from said first cytokine.

36. The method of claim 34, wherein said cytokine is selected from the group consisting of GM-CSF, IL-4, C-KIT, Steel factor, TGF-β, TNF-α and FLT3 ligand.

37. The method of claim 34, wherein said cytokine is administered as a gene encoded by said expression construct.

38. A method for treating a pathogen-induced disease in a subject comprising the steps of:
(i) identifying a subject with a pathogen-induced disease characterized by alteration or increased expression of a pathogen gene product in at least some of the pathogen-induced cells in said patient;
(ii) intradermally administering to said subject an expression construct comprising a pathogen gene under the control of a promoter operable in eukaryotic dendritic cells;
(ii) infecting said dendritic cells with an adenoviral vector comprising a pathogen gene product under the control a promoter operable in eukaryotic cells; and
(iii) administering the adenovirus-infected dendritic cells to said subject,
whereby said pathogen gene product is expressed by dendritic cells and presented to immune effector cells, thereby stimulating an anti-pathogen gene product response.

39. The method of claim 38, wherein said dendritic cells are obtained from peripheral blood progenitor cells.

40. The method of claim 38, further comprising multiple administrations of adenovirus-infected dendritic cells.

41. The method of claim 38, wherein said pathogen is selected from the group consisting of bacterium, virus, fungus, parasitic worm, amoebae and mycoplasma.

42. The method of claim 41, wherein said bacterium is selected from the group consisting of richettsia, listeria and histolytica.

43. The method of claim 41, wherein said virus is selected from the group consisting of HIV, HBV, HCV, HSV, HPV, EBV and CMV.

44. The method of claim 41, wherein said fungus is selected from the group consisting of hitoplasma, coccidis, immitis, aspargillus, actinomyces, blastomyces, candidia and streptomyces.

45. The method of claim 38, wherein said expression construct is a viral vector.

46. The method of claim 45, wherein said viral vector is an adenoviral vector, a retroviral vector, a vaccinia viral vector, an adeno-associated viral vector, a polyoma viral vector, an alphavirus vector, or a herpesviral vector.

47. The method of claim 46, wherein said viral vector is an adenoviral vector.

48. The method of claim 47, wherein said adenoviral vector is replication-defective.

49. The method of claim 48, wherein the replication defect is a deletion in the E1 region of the virus.

50. The method of claim 49, wherein the deletion maps to the E1B region of the virus.

51. The method of claim 50, wherein the deletion encompasses the entire E1B region of the virus.

52. The method of claim 51, wherein the deletion encompasses the entire E1 region of the virus.

53. The method of claim 38, wherein said promoter is selected from the group consisting of CMV IE, human or murine dectin-1, human or murine dectin-2, human CD11c, mammalian F4/80 and human or murine MHC class II.

54. The method of claim 53, wherein said promoter is CMV IE.

55. The method of claim 38, wherein said expression vector further comprises a polyadenylation signal.

56. The method of claim 38, wherein intradermal administration is by injection of the expression construct.

57. The method of claim 56, further comprising multiple injections.

58. The method of claim 56, wherein the injection is performed local to a pathogen-induced disease site.

59. The method of claim 56, wherein the injection is performed regional to a pathogen-induced disease site.

60. The method of claim 56, wherein the injection is performed distal to a pathogen-induced disease site.
